(19) United States
(12) Patent Application Publication
(10) Pub. No.: US 2012/0021925 A1
(43) Pub. Date: Jan. 26, 2012

(54) DIAGNOSTIC ASSAYS FOR PROSTATE CANCER USING PSP94 AND PSA BIOMARKERS

(75) Inventors: Katrin Atnikov, Winnipeg (CA); Douglas Barker, Winnipeg (CA); Yilan Zhang, Winnipeg (CA); Stephen Frost, Gurnee, IL (US)

(73) Assignee: MIRACULINS, INC., Winnipeg (CA)

(21) Appl. No.: 13/144,662

(22) PCT Filed: Jan. 19, 2010
(86) PCT No.: PCT/CA10/00078

§ 371 Date: Oct. 5, 2011

Related U.S. Application Data
(60) Provisional application No. 61/145,671, filed on Jan. 19, 2009.
(60) Provisional application No. 61/151,308, filed on Feb. 10, 2009.

(51) Int. Cl. C40B 30/00; H01J 49/40; C12Q 1/37; H01J 49/26; G01N 33/577; G01N 21/64

(52) U.S. Cl. 506/7; 436/501; 435/7.1; 435/7.9; 435/23; 435/7.92; 250/282

(57) ABSTRACT

The application refers to a method for diagnosing prostate cancer through the measurement of the combination of PSP94 (β-microseminoprotein) and F/T PSA biomarkers, the method using urine samples to measure PSP94 and serum samples to analyse F/T PSA. In one embodiment, the PSP94 is standardized to creatinine. Also based on the measurement of PSP94 and F/T PSA are provided methods and kits for (a) diagnosing aggressive prostate cancer; (b) differential diagnosis; and (c) diagnosing the progression of prostate cancer.

### FIELD OF THE INVENTION

The present invention relates diagnosing prostate diseases. More particularly, the present invention includes a method for differential diagnosis of prostate cancer from a non-malignant disease of the prostate and/or from a healthy prostate.

### BACKGROUND

Prostate cancer is one of the most common cancers to afflict men in western countries. In North America the incidence rate for prostate cancer in males is an estimated 166.7 per 100,000 per year, which accounted for an estimated 33% of all newly reported cancers in men in 2005 (American Cancer Society 2005). The Canadian Cancer Society indicates that one in 7 men will develop prostate cancer, mostly after age 70 (Canadian Cancer Society 2005). In 2005, American Cancer Society and Canadian Cancer Society estimated the mortality rate for this disease to be 20% (American Cancer Society 2005; Canadian Cancer Society 2005).

The current standard screening method for prostate cancer is the Prostate Specific Antigen (PSA) test, which can take the form of total PSA measurements, free:total PSA ratios, and PSA velocities (change in PSA levels over time) (Egawa et al. 1997; Djavan et al. 1999). An individual has typically been characterized as having an elevated risk for prostate cancer with a PSA level above 4.0 ng/mL (Gann et al. 1995). This can be refined to account for a number of factors, such as PSA levels increasing naturally with age (Oesterling et al. 1994). Clinicians must rely on complementary diagnostic tools because PSA screening is an imperfect means of diagnosis, is not indicative of pathological stage (Beduschi and Oesterling 1997; Erdem et al. 2002-2003), and has poor specificity. The result is healthy patients being subjected to unnecessary testing and an increased financial and emotional toll of prostate cancer diagnosis. The primary diagnostic tools used in addition to PSA testing are the digital-rectal exam (DRE) and prostate biopsy. DREs are performed routinely in conjunction with PSA tests and biopsies to improve the accuracy of diagnosis (Scattoni et al. 2003). Prostate biopsies are the means of ultimate confirmation of diagnosis, but have significant complication rates (Rodriguez and Terris 1998). The U.S. Preventative Services Task Force does not recommend the PSA test for routine screening. Despite the known shortcomings of PSA testing and significant amounts of research, there has been little improvement in the state of the prostate disease diagnostics. Thus, there is an unmet need for more accurate prostate disease diagnostics, particularly prostate cancer.

Prostate Secretory Protein (PSP94), also known as beta-microseminoprotein, or inhibin-like peptide is a basic 94 amino acid protein with a MW of 10,704 (Seidah et al, FEBS Lett, 1984, 175(2): 349-55). PSP94 is generated from a 114 amino acid precursor whose DNA sequence is located on chromosome 10 (Dube et al, J Androl, 1987, 8(3): 182-9). Purified PSP94 isolated from seminal fluid migrates between 13-16 kDa on a polyacrylimide gel (Dube et al, 1987), and the difference in molecular weight is not due to glycosylation but due to the basic nature of the protein (Seidah et al, 1984).

PSP94 is found in high concentration in the epithelial cells of the prostate (Brar et al, J Androl, 1988, 9(4): 253-260). A 31 amino acid cleavage product of PSP94 found in seminal fluid demonstrates the ability to inhibit FSH release (Ramasharma et al, Science, 1984, 223(4641): 1199-1202). Later, PSP94 was examined in serum and urine for its potential to be a cancer biomarker. Results from Kaighn et. al. (Kaighn et al, Invest Urol, 1987, 17(1): 16-23) demonstrated that PSP94 was not detectable in PC-3 cell line from human prostatic carcinomas. Furthermore, PSP94 in urine was decreased in men with late stage tumors using 24 hr collection (Teni et al, Cancer Letters, 1988, 43(1-2): 9-14; Teni et al, Clin Chem, 1989, 35(7): 1376-9; Trembley et al, Prostate, 1987, 10(3): 235-43).

### SUMMARY OF THE INVENTION

Recently, we identified a peak at 10750 M/Z by mass spectrometry that decreased in the urine of patients with prostate cancer. The peak corresponded to PSP94. We then developed an immunoassay to measure PSP94 in urine to develop a commercial assay to help minimize the number of prostate biopsies for men with PSA values between 2.5 and 10 ng/mL with a negative DRE.

An aspect of the present invention relates to methods for differential diagnosis of prostate cancer or non-malignant disease of the prostate by detecting PSP94 and PSA and determining free:total PSA (F/T PSA) within a test sample of a given subject, comparing results with samples from healthy subjects, subjects having precancerous prostatic lesion, subjects with non-malignant disease of the prostate, subjects with localized cancer of the prostate, subjects with metastasised cancer of the prostate, and/or subjects with an acute or a chronic inflammation of prostatic tissue, wherein comparison allows for differential diagnosis of a subject as healthy, having a precancerous prostatic lesion, having non-malignant disease of the prostate, having localized prostate cancer, having a metastasised prostate cancer or having an acute or chronic inflammation of prostatic tissue. In an embodiment, the subject does not have hypertension.

One aspect of the invention includes a method for diagnosing prostate cancer in a subject comprising detecting a quantity, presence, or absence of PSP94 and F/T PSA in a biological sample; and classifying said subject as having or not having prostate cancer, based on said quantity, presence or absence of PSP94 and F/T PSA. In one embodiment, the step of classifying said subject comprises comparing the quantity, presence, or absence of PSP94 and F/T PSA with a reference value indicative of a prostate cancer. In an embodiment, the subject does not have hypertension.

A further aspect of the invention includes a method for differential diagnosis of prostate cancer and non-malignant disease of the prostate in a subject, comprising detecting a quantity, presence or absence of PSP94 and F/T PSA in a biological sample and classifying said subject as having prostate cancer, non-malignant disease of the prostate, or as healthy, based on the quantity, presence or absence of PSP94 and F/T PSA in said biological sample. In one embodiment, the step of classifying said subject comprises comparing a quantity, presence, or absence of PSP94 and F/T PSA with a reference value indicative of prostate cancer and a reference value indicative of a non-malignant disease of the prostate. In an embodiment, the subject does not have hypertension. A further aspect of the invention includes a method for differential diagnosis of healthy, non-malignant disease of the prostate, precancerous prostatic lesion, localized cancer of the prostate, metastasised cancer of the prostate, and acute or chronic inflammation of prostatic tissue in a subject, comprising detecting a quantity, presence or absence of PSP94 and F/T PSA in a biological sample and classifying said subject as having non-malignant disease of the prostate, precancerous prostate lesion, localized cancer of the prostate, metastasised cancer of the prostate, and/or acute or chronic inflammation of prostatic tissue, or as healthy, based on the quantity, presence or absence of PSP94 and F/T PSA in said biological sample. In one embodiment, a step of classifying said subject comprises comparing a quantity, presence or absence of PSP94 and F/T PSA with a reference value indicative of healthy, non-malignant disease of the prostate, precancerous prostate lesion, localized cancer of the prostate, metastasised cancer of the prostate, acute inflammation of prostatic tissue or chronic inflammation of prostatic tissue. In an embodiment, the subject does not have hypertension.

In a further embodiment, a method for diagnosing a prostate cancer in a subject or the method for differential diagnosis of healthy, non-malignant disease of the prostate, precancerous prostatic lesion, localized cancer of the prostate, metastasised cancer of the prostate, and acute or chronic inflammation of prostatic tissue in a subject, PSP94 and F/T PSA are used to classify a subject by: (a) contacting a biological sample with a biologically active surface, (b) allowing the PSP94 and PSA within the biological sample to bind to the biologically active surface; (c) detecting the bound PSP94 and PSA, and determining F/T PSA, using a detection method, wherein the detection method generates mass profiles of the biological sample; (d) transforming the information obtained in c) into a computer readable form; and (e) comparing the information in d) with a database containing mass profiles from subjects whose classification is known; wherein the comparison allows for the differential diagnosis and classification of a subject. In an embodiment, the subject does not have hypertension. In another embodiment, the database comprises mass profiles from subjects whose classification is known, wherein subjects with hypertension are excluded.

In a further aspect of the invention, expression of PSP94 as used to determine diagnosis may be determined in conjunction with and standardized to creatinine levels.

In a further aspect diagnosis includes differential diagnosis.

An aspect of the invention includes a method for determining aggressiveness or non-aggressiveness of prostate cancer, the method comprising comparing 1) quantity of PSP94 and F/T PSA, in a subject's test sample; and 2) quantity of PSP94 and F/T PSA, in a control/benign sample. A difference in the quantity in the subject's sample and the quantity in the control/benign sample is an indication that prostate cancer is aggressive or non-aggressive. In an embodiment, the subject does not have hypertension.

In a further aspect of the invention aggressiveness of prostate cancer may be determined when the PSA score is 2.5-10 ng/mL.

In a further aspect of the invention, expression of PSP94 as used to determine aggressiveness or non-aggressiveness may be determined in conjunction with and standardized to creatinine levels.

An aspect of the present invention relates to methods for evaluating a prognosis of prostate cancer in a subject. The methods comprise detecting a quantity of PSP94 and F/T PSA in a test sample; and classifying the progression of cancer. The present method permits differentiation of prostate cancer subjects with a good prognosis (high probability of recovery, becoming disease free) from subjects with a bad prognosis (low probability of recovery, cancer reoccurrence, metastasis). In an embodiment, the subject does not have hypertension.

In a further aspect of the invention, expression of PSP94 as used to determine prognosis may be determined in conjunction with and standardized to creatinine levels.

In a further embodiment of the methods of the invention, a database is generated by (a) obtaining reference biological samples from subjects having known classification; (b) contacting the reference biological samples in (a) with a biologically active surface, (c) allowing PSP94 and F/T PSA within the reference biological samples to bind to the biologically active surface, (d) detecting bound PSP94 and PSA, and determining F/T PSA, using a detection method, wherein the detection method generates mass profiles of the reference biological samples, (e) transforming the mass profiles into a computer-readable form, and (f) applying a mathematical algorithm to classify the mass profiles in d) into desired classification groups. In an embodiment, the database excludes data compiled from subjects with hypertension.

In a further aspect of the invention, expression of PSP94 as used to generate a database may be determined in conjunction with and standardized to creatinine levels. In a further embodiment of the methods of the invention, a quantity, presence, or absence of PSP94 and F/T PSA are detected in a biological sample obtained from a subject by mass spectrometry. A method of mass spectrometry can be matrix-assisted laser desorption ionization/time of flight (MALDI-TOF), surface enhanced laser desorption ionisation/time of flight (SELDI-TOF), liquid chromatography, MS-MS, or ESI-MS. In an embodiment, the subject does not have hypertension.

In a further embodiment of the methods of the invention, a quantity, presence, or absence of PSP94 and F/T PSA are detected or quantified in a biological sample obtained from the subject utilizing an antibody to said biomarker.

In a further embodiment of the methods of the invention, a quantity, presence, or absence of PSP94 and F/T PSA are detected or quantified in a biological sample obtained from the subject through the use of an ELISA assay.

In a further embodiment of the methods of the invention, a quantity, presence, or absence of PSP94 and F/T PSA are detected or quantified in a biological sample obtained from the subject through the use of a BioPlex Immunoassay (Bio-Rad Laboratories, Hercules, Calif.).

In a further embodiment of the methods of the invention, a quantity, presence, or absence of PSP94 and F/T PSA are detected or quantified through a use of a biochip.

In a further embodiment of the invention, a quantity, presence, or absence of PSP94 and F/T PSA are detected or quantified in an automated system.

In a further embodiment of the invention, a quantity, presence or absence of PSP94 may be determined in conjunction with and standardized to creatinine levels.

In a further embodiment of the invention, a subject is a mammal. The subject may be a human.

In a further embodiment of the invention, a test or biological sample used according to the invention may be blood, blood serum, blood plasma, urine, semen, seminal fluid, seminal plasma, prostatic fluid, pre-ejaculatory fluid (Cowper's fluid), excreta, tears, saliva, sweat, bile, biopsy, ascites, cerebrospinal fluid, lymph, or tissue extract origin. In a further embodiment of the methods of the invention, the test and/or biological samples are urine, semen, seminal fluid, seminal plasma, prostatic fluid, pre-ejaculatory fluid (Cowper's fluid) samples, and are isolated from subjects of mammalian origin, preferably of human origin. In a still further embodiment of the invention, the test and/or biological samples are blood, blood serum, plasma and/or urine.

In a further embodiment of the invention, a biologically active surface comprises an adsorbent comprising silicon dioxide molecules.

A further aspect of the invention includes a kit for diagnosing prostate disease within a subject comprising: a biologically active surface comprising an adsorbent, binding solutions, and instructions to use the kit, wherein the instructions outline a method for diagnosis of a prostate cancer in a subject according to the invention or a method for the differential diagnosis of healthy, non-malignant disease of the prostate, precancerous prostatic lesion, localized cancer of the prostate, metastasised cancer of the prostate, and acute or chronic inflammation of prostatic tissue in a subject according to the invention. In an embodiment, the subject does not have hypertension.

In an embodiment of the invention, a kit comprises a biologically active surface comprising an adsorbent comprised of silicon dioxide molecules.

In an embodiment of the invention, a kit comprises a biologically active surface comprising an adsorbent comprising antibodies specific to PSP94 and PSA.

In a further aspect of the invention, expression of PSP94 as determined by the use of a kit may be determined in conjunction with and standardized to creatinine levels.

A further aspect of the invention includes a method for in vitro diagnosis of a prostate cancer in a subject comprising detecting PSP94 and F/T PSA in a biological sample by: (a) contacting a biological sample from a subject with one or more binding molecule specific for PSP94 and PSA and (b) detecting a quantity, presence or absence of PSP94 and PSA, and determining F/T PSA, in the sample, wherein a quantity, presence or absence of PSP94 and F/T PSA allows for diagnosis of the subject as healthy or having prostate cancer. In an embodiment, the subject does not have hypertension.

A further aspect of the invention includes a method for in vitro differential diagnosis of prostate cancer and non-malignant disease of the prostate in a subject, comprising detecting PSP94 and F/T PSA in a biological sample: (a) contacting a biological sample with a binding molecule specific for PSP94 and PSA; and (b) detecting a quantity, presence or absence of PSP94 and PSA, and determining F/T PSA, in the sample, wherein the quantity, presence or absence of PSP94 and F/T PSA allows for the differential diagnosis of the subject as having prostate cancer, and/or having a non-malignant disease of the prostate, or as being healthy. In an embodiment, the subject does not have hypertension.

In an embodiment according to the invention for in vitro diagnosis of prostate cancer in a subject, for in vitro differential diagnosis of prostate cancer and non-malignant disease of the prostate in a subject, or for in vitro differential diagnosis of healthy, prostate cancer, non-malignant disease of the prostate, precancerous prostatic lesion, localized cancer of the prostate, metastasised cancer of the prostate, and acute or chronic inflammation of prostatic tissue in a subject, detection is performed by an immunosorbent assay. In an embodiment, the subject does not have hypertension.

In a further aspect of the invention, expression of PSP94 as determined for in vitro diagnoses may be determined in conjunction with and standardized to creatinine levels.

A further aspect of the invention comprises a kit for diagnosis of a prostate disease within a subject comprising a binding solution, one or more binding molecule(s), a detection substrate, and instructions, wherein the instructions outline a method according to the invention for in vitro diagnosis of prostate cancer in a subject, for in vitro differential diagnosis of prostate cancer and non-malignant disease of the prostate in a subject, or for in vitro differential diagnosis of healthy, prostate cancer, non-malignant disease of the prostate, precancerous prostatic lesion, localized cancer of the prostate, metastasised cancer of the prostate, and acute or chronic inflammation of prostatic tissue in a subject. In an embodiment, the subject does not have hypertension.

In a further aspect of the invention, expression of PSP94, as detected by the use of a kit as disclosed herein, may be determined in conjunction with and standardized to creatinine levels.

A further aspect of the invention comprises a use of PSP94 and F/T PSA for differential diagnosis of non-malignant disease of the prostate, precancerous prostatic lesion, localized cancer of the prostate, metastasised cancer of the prostate or acute or chronic inflammation of prostatic tissue.

A further aspect of the invention comprises a use of the detection or quantification of PSP94 and F/T PSA in a biological sample from a subject for determination of whether the subject has prostate cancer. In an embodiment, the subject does not have hypertension.

A further aspect of the invention comprises a use of the detection or quantification of PSP94 and F/T PSA in a biological sample from a subject for determination of whether the subject has non-malignant disease of the prostate. In an embodiment, the subject does not have hypertension.

A further aspect of the invention comprises a use of the detection or quantification of PSP94 and F/T PSA in a biological sample from a subject for determination of whether the subject has benign prostate disease, precancerous prostatic lesions, localized cancer of the prostate, metastasised cancer of the prostate, or acute or chronic inflammation of the prostate. In an embodiment, the subject does not have hypertension.

In a further aspect of the invention, detection and quantification of PSP94 may be determined in conjunction with and standardized to creatinine levels.

A further aspect of the invention comprises a database containing a plurality of database entries useful in diagnosing subjects as having, or not having, prostate cancer, comprising: (a) a categorization of each database entry as either characteristic of having, or not having prostate cancer; and (b) characterization of each database entry as either having, or not having, or having in a certain quantity, PSP94 and F/T PSA. In an embodiment, the database entries exclude any data from subjects with hypertension.

In an embodiment of the invention, a database can further include a characterization of each database entry as either having, or not having, or having in a certain quantity of PSP94 and F/T PSA.

A further aspect of the invention comprises a database generated by: (a) obtaining reference biological samples from subjects known to have, and patients known not to have, prostate cancer; (b) contacting the reference biological samples in (a) with a biologically active surface; (c) allowing PSP94 and PSA within the reference biological samples to bind to the biologically active surface; (d) detecting bound PSP94 and PSA, and determining F/T PSA, using a detection method wherein the detection method generates mass profiles of the reference biological samples; (e) transforming the mass profiles into a computer readable form; and (f) applying a mathematical algorithm to classify the mass profiles in (d) as specific for healthy subjects or subjects having prostate cancer. In an embodiment of the database, reference biological samples are obtained from subjects without hypertension.

In a further aspect of the invention, a database with reference values may contain values of PSP94 which may be determined in conjunction with and standardized to creatinine levels.

A further aspect of the invention includes memory for storing data for access by an application program being executed on a data processing system for diagnosing a prostate cancer or a non-malignant prostate disease, comprising a data structure stored in the memory, the data structure including information resident in a database used by the application program and including one or more reference values stored in the memory having a plurality of mass profiles associated with PSP94 and F/T PSA, with or without standardization to creatinine, previously defined as being characteristic of a prostate cancer or a non-malignant disease of the prostate; wherein each of the mass profiles has been transformed into a computer readable form.

A further aspect of the invention comprises a use of PSP94 and F/T PSA to detect prostate cancer.

In a further aspect of the invention, use of PSP94 can be in conjunction with and standardized to creatinine levels.

A further aspect of the invention includes a method of identifying a molecular entity that inhibits or promotes an activity of PSP94 and F/T PSA according to the invention comprising: (a) selecting a control animal having PSP94 and F/T PSA and a test animal having PSP94 and F/T PSA; (b) treating the test animal using the molecular entity or a library of molecular entities, under conditions to allow specific binding and/or interaction, and (c) determining a relative quantity of PSP94 and F/T PSA, as between the control animal and the test animal. Activity of PSP94 can be determined in conjunction with and standardized to creatinine.

In an embodiment of the invention, animals are mammals. Mammals may be rats, mice, or primates.

A further aspect of the invention includes a method of identifying a molecular entity that inhibits or promotes an activity of PSP94 and F/T PSA comprising: (a) selecting a host cell expressing PSP94 and PSA; (b) cloning the host cell and separating the clones into a test group and a control group; (c) treating the test group using the molecular entity or a library of molecular entities under conditions to allow specific binding and/or interaction, and (d) determining a relative quantity of PSP94 and F/T PSA, as between the test group and the control group.

A further aspect of the invention includes a method for identifying a molecular entity that inhibits or promotes an activity of PSP94 and F/T PSA comprising: (a) selecting a test group having a host cell expressing PSP94 and F/T PSA and a control group; (b) treating the test group using the molecular entity or a library of molecular entities; and (c) determining a relative quantity of PSP94 and F/T PSA, as between the test group and the control group. Activity of PSP94 can be determined in conjunction with and standardized to creatinine.

In an embodiment of the invention, a host cell is a neoplastic or cancer cell.

In an embodiment of any of the methods according to the invention for identifying a molecular entity that inhibits or promotes an activity of PSP94 and F/T PSA, a library of molecular entities can be nucleotides, oligonucleotides, polynucleotides, amino acids, peptides, polypeptides, proteins, antibodies, immunoglobulins, small organic molecules, pharmaceutical agents, agonists, antagonists, derivatives, and/or combinations thereof.

In a further aspect of the invention, the activity of PSP94 can be determined in conjunction with and standardized to creatinine.

A further aspect of the invention includes a composition for treating a prostate disease comprising a molecular entity, which modulates PSP94 and F/T PSA and a pharmaceutically acceptable carrier.

In a further aspect of the invention, the modulation of PSP94 can be determined in conjunction with and standardized to creatinine.

An embodiment of the invention includes a composition for treating a prostate disease selected from the group consisting of prostate cancer and non-malignant disease of the prostate.

A further embodiment includes a composition for treating a prostate disease selected from the group consisting of non-malignant disease of the prostate, precancerous prostatic lesion, localized cancer of the prostate, metastasised cancer of the prostate, and acute or chronic inflammation of prostatic tissue.

A further embodiment of the invention includes a composition comprising a molecular entity that can be polynucleotides, amino acids, polypeptides, small organic molecules, pharmaceutical agents, or combinations thereof. The polypeptides can be antibodies, agonists, antagonists, derivatives, or combinations thereof.

A further aspect of the invention includes a composition for treating prostate disease comprising a molecular entity identified by any one of the methods of invention for identifying a molecular entity, which inhibits or promotes the activity of PSP94 and F/T PSA and a pharmaceutically acceptable carrier. Activity of PSP94 can be determined in conjunction with and standardized to creatinine.

In an embodiment of the invention, a composition comprises a molecular entity that is comprised of polynucleotides, amino acids, peptides, polypeptides, proteins, small organic molecules, pharmaceutical agents, agonists, antagonists, derivatives or combinations thereof.

A further aspect of the invention includes a use of any composition according to the invention for treating a prostate disease. Prostate disease may be prostate cancer and non-malignant disease of the prostate. The prostate disease may be is selected from the group consisting of non-malignant disease of the prostate, precancerous prostatic lesion, localized cancer of the prostate, metastasised cancer of the prostate, and acute or chronic inflammation of prostatic tissue.

### BRIEF DESCRIPTION OF THE DRAWINGS

FIG. 1 is a graphical depiction of a typical calibration curve.

FIG. 2 is a graphical depiction of spike recovery data.

FIG. 3 is a ROC curve generated for PSP94 concentration, F/T PSA and the combination of PSP94 and F/T PSA.

FIG. 4 is a ROC curve generated for PSP94/creatinine, F/T PSA and the combination of PSP94/Creatinine and F/T PSA.

### DETAILED DESCRIPTION OF THE INVENTION

The term “biomolecule” refers to a molecule that is produced by a cell or tissue in an organism. Such molecules include, but are not limited to, molecules comprising polynucleotides, amino acids, peptides, polypeptides, proteins, sugars, carbohydrates, fatty acids, lipids, steroids, and combinations thereof (e.g., glycoproteins, ribonucleoproteins, lipoproteins). Furthermore, the terms “nucleotide”, “oligonucleotide” or polynucleotide” refer to DNA or RNA of genomic or synthetic origin which may be single-stranded or double-stranded and may represent the sense or the antisense strand. Included as part of the definition of “oligonucleotide” or “polynucleotide” are peptide polynucleotide sequences (i.e. peptide nucleic acids; PNAs), or any DNA-like or RNA-like material (e.g. morpholinos, ribozymes).

“Polypeptide” refers to a peptide or protein containing two or more amino acids linked by peptide bonds, and includes peptides, oligomers, proteins, and the like. Polypeptides can contain natural, modified, or synthetic amino acids. Polypeptides can also be modified naturally, such as by post-translational processing, or chemically, such as amidation acylation, cross-linking, glycosylation, pegylation, and the like.

The term “antibody” is used in the broadest sense and specifically includes monoclonal antibodies (including full length monoclonal antibodies), multispecific antibodies (e.g., bispecific antibodies), and antibody fragments that exhibit a desired biological activity or function. Antibodies can be chimeric, humanized, or mammalian, including mouse or human. Antibodies can also be an antibody fragment.

“Antibody fragments” comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments. “Functional fragments” substantially retain binding to an antigen of the full length antibody, and retain a biological activity.

The term “molecular entity” refers to any defined inorganic or organic molecule that is either naturally occurring or is produced synthetically. Such molecules include, but are not limited to, biomolecules as described above, simple and complex molecules, acids and alkalis, alcohols, aldehydes, arenas, amides, amines, esters, ethers, ketones, metals, salts, and derivatives of any of the aforementioned molecules.

The term “fragment” refers to a portion of a polynucleotide or polypeptide sequence that comprises at least 15 consecutive nucleotides or 5 consecutive amino acid residues, respectively. Furthermore, these “fragments” typically retain the biological activity and/or some functional characteristics of the parent polypeptide e.g. antigenicity or structural domain characteristics.

The term “prostatic secretory protein 94” or “PSP94” refers to a 94 amino acid protein secreted by the prostate that functions as a tumor suppressor. PSP94 is the mature protein that is amino acid residues 1 to 94 of the full-length 114 amino acid protein of SEQ ID NO:1. The terms “Prostate Secretory protein 94”, “PSP94”, “Prostate Secreted Seminal Plasma Protein”, “Seminal Plasma Beta-Inhibin”, “Immunoglobulin-binding factor”, “IGBF”, and “PN44” are used interchangeably herein.

The term “free PSA” refers to PSA that is unbound or not bound to another entity.

The term “bound PSA” refers to PSA that is bound to another entity.

The term “total PSA” refers to the sum of free PSA and bound PSA.

The term “free:total PSA” or “F/T PSA” is the ratio of unbound PSA to total PSA.

The terms “biological sample” and “test sample” are used interchangeably and refer to all biological fluids and excretions isolated from any given subject. In the context of the invention such samples include, but are not limited to, blood, blood serum, blood plasma, urine, semen, seminal fluid, seminal plasma, prostatic fluid, pre-ejaculatory fluid (Cowper's fluid), excreta, tears, saliva, sweat, biopsy, ascites, cerebrospinal fluid, lymph, marrow, hair or tissue extract samples such as homogenized tissue, and cellular extracts, and combinations thereof. Tissue samples include samples of tumors.

The term “host cell” refers to a cell that has been transformed or transfected, or is capable of transformation or transfection by an exogenous polynucleotide sequence. It is understood that such terms refer not only to the particular subject cell but also to the progeny or potential progeny of such a cell. Since certain modifications may occur in succeeding generations due to either mutation or environmental influences, such progeny may not, in fact, be identical to the parent cell, but are still included within the scope of the term as used herein.

The term “specific binding” refers to an interaction between two biomolecules that occurs under specific conditions. The binding of two biomolecules is considered to be specific when the interaction between said molecules is substantial. In the context of the invention, a binding reaction is considered substantial when the signal of the peak representing the biomolecule is at least twice that of the signal arising from the coincidental detection of non-biomolecule associated ions in approximately the same mass range, which is the peak as a signal to noise ratio of at least two. Moreover, the phrase “specific conditions” refers to reaction conditions that permit, enable, or facilitate the binding of said molecules such as pH, salt, detergent and other conditions known to those skilled in the art.

The term “interaction” relates to the direct or indirect binding or alteration of biological activity of a biomolecule.

The term “differential diagnosis” refers to a diagnostic decision between healthy and different disease states, including various stages of a specific disease. A subject is diagnosed as healthy or to be suffering from a specific disease, or a specific stage of a disease based on a set of hypotheses that allow for the distinction between healthy and one or more stages of the disease. A choice between healthy and one or more stages of disease depends on a significant difference between each hypothesis. Under the same principle, a “differential diagnosis” may also refer to a diagnostic decision between one disease type as compared to another (e.g., prostate cancer versus a non-malignant disease of the prostate).

The term “prostate cancer” refers to a malignant neoplasm of the prostate within a given subject, wherein the neoplasm is of epithelial origin and is also referred to as a carcinoma of the prostate. Prostate cancer can be defined according to its type, stage and/or grade. Typical staging systems include the Jewett-Whitmore system and the TNM system (the system adopted by the American Joint Committee on Cancer and the International Union Against Cancer). A typical grading system is the Gleason Score which is a measure of tumour aggressiveness based on pathological examination of tissue biopsy). The term “prostate cancer”, when used without qualification, includes both localized and metastasised prostate cancer. The term “prostate cancer” can be qualified by the terms “localized” or “metastasised” to differentiate between different types of tumour as those words are defined herein. The terms “prostate cancer” and “malignant disease of the prostate” are used interchangeably herein.

The terms “neoplasm” or “tumour” may be used interchangeably and refer to an abnormal mass of tissue wherein the growth of the mass surpasses and is not coordinated with the growth of normal tissue. A neoplasm or tumour may be defined as “benign” or “malignant” depending on the following characteristics: degree of cellular differentiation including morphology and functionality, rate of growth, local invasion and metastasis. A “benign” neoplasm is generally well differentiated, has characteristically slower growth than a malignant neoplasm and remains localised to the site of origin. In addition a benign neoplasm does not have the capacity to infiltrate, invade or metastasise to distant sites. A “malignant” neoplasm is generally poorly differentiated (anaplasia), has characteristically rapid growth accompanied by progressive infiltration, invasion and destruction of the surrounding tissue. Furthermore, a malignant neoplasm has to capacity to metastasise to distant sites.

The term “differentiation” refers to the extent to which parenchymal cells resemble comparable normal cells both morphologically and functionally.

The term “metastasis” refers to spread or migration of cancerous cells from a primary (original) tumour to another organ or tissue, and is typically identifiable by the presence of a “secondary tumour” or “secondary cell mass” of the tissue type of the primary (original) tumour and not of that of the organ or tissue in which the secondary (metastatic) tumour is located. For example, a prostate cancer that has migrated to bone is said to be metastasised prostate cancer, and consists of cancerous prostate cancer cells in the prostate as well as cancerous prostate cancer cells growing in bone tissue.

The terms “a non-malignant disease of the prostate”, “non-prostate cancer state” and “benign prostatic disease” may be used interchangeably and refer to a disease state of the prostate that has not been classified as prostate cancer according to specific diagnostic methods including but not limited to rectal palpitation, PSA scoring, transrectal ultrasonography and tissue biopsy. Such diseases include, but are not limited to, an inflammation of prostatic tissue (i.e., chronic bacterial prostatitis, acute bacterial prostatitis, chronic abacterial prostatitis) and benign prostate hyperplasia.

The term “healthy” refers to an absence of any malignant or non-malignant disease of the prostate; thus, a “healthy individual” may have other diseases or conditions that would normally not be considered “healthy”. A “healthy” individual demonstrates an absence of any malignant or non-malignant disease of the prostate.

The term “pre-cancerous lesion of the prostate” or “precancerous prostate lesion” refers to a biological change within the prostate such that it becomes susceptible to the development of a malignant neoplasm. More specifically, a pre-cancerous lesion of the prostate is a preliminary stage of a prostate cancer. Causes of a pre-cancerous lesion may include, but are not limited to, genetic predisposition and exposure to cancer-causing agents (carcinogens); such cancer causing agents include agents that cause genetic damage and induce neoplastic transformation of a cell.

The term “neoplastic transformation of a cell” refers to an alteration in normal cell physiology and includes, but is not limited to, self-sufficiency in growth signals, insensitivity to growth-inhibitory (anti-growth) signals, evasion of programmed cell death, limitless replicative potential, sustained angiogenesis, and tissue invasion and metastasis.

The term “differentially present” refers to differences in the quantity of a biomolecule present in samples taken from prostate cancer patients as compared to samples taken from subjects having a non-malignant disease of the prostate or healthy subjects. Furthermore, a biomolecule is differentially present between two samples if the quantity of said biomolecule in one sample population is significantly different (defined statistically) from the quantity of said biomolecule in another sample population. For example, a given biomolecule may be present at elevated, decreased, or absent levels in samples of taken from subjects having prostate cancer compared to those taken from subjects who do not have a prostate cancer.

The term “biological activity” may be used interchangeably with the terms “biologically active”, “bioactivity” or “activity” and, for the purposes herein, means an effector or antigenic function that is directly or indirectly performed by a biomarker of the invention (whether in its native or denatured conformation), derivative or fragment thereof. Effector functions include phosphorylation (kinase activity) or activation of other molecules, induction of differentiation, mitogenic or growth promoting activity, signal transduction, immune modulation, DNA regulatory functions and the like. Antigenic functions include possession of an epitope or antigenic site that is capable of cross-reacting with antibodies raised against a naturally occurring or denatured biomarker of the invention, derivative or fragment thereof. Accordingly, a biological activity of such a protein can be that it functions as regulator of a signalling pathway of a target cell. Such a signalling pathway can, for example, modulate cell differentiation, proliferation and/or migration of such a cell, as well as tissue invasion, tumour development and/or metastasis. A target cell according to the invention can be a neoplastic or cancer cell.

The terms “neoplastic cell” and “neoplastic tissue” refer to a cell or tissue, respectively, that has undergone significant cellular changes (transformation). Such cellular changes are manifested by an escape from specific control mechanisms, increased growth potential, alteration in the cell surface, karyotypic abnormalities, morphological and biochemical deviations from the norm, and other attributes conferring the ability to invade, metastasise and kill.

The term “diagnostic assay” can be used interchangeably with “diagnostic method” and refers to the detection of the presence or nature of a pathologic condition. Diagnostic assays differ in their sensitivity and specificity, and their relative usefulness as a diagnostic tool can be measured using ROC-AUC statistics.

Herein, the term “true positives” refers to those subjects having a localized or a metastasised cancer of the prostate or a benign prostate disease, a precancerous prostatic lesion, or an acute or a chronic inflammation of prostatic tissue and who are categorized as such by the diagnostic assay. Depending on context, the term “true positives” may also refer to those subjects having either prostate cancer or a non-malignant disease of the prostate, who are categorized as such by the diagnostic assay.

Herein, the term “false negatives” refers to those subjects having either a localized or a metastasised cancer of the prostate, a benign prostate disease, a precancerous prostatic lesion, or an acute or a chronic inflammation of prostatic tissue and who are not categorized as such by the diagnostic assay. Depending on context, the term “false negatives” may also refer to those subjects having either prostate cancer or a non-malignant disease of the prostate and who are not categorized as such by the diagnostic assay.

Herein, the term “true negatives” refers to those subjects who do not have a localized or a metastasised cancer of the prostate, a benign prostate disease, a precancerous prostatic lesion, or an acute or a chronic inflammation of prostatic tissue and who are categorized as such by the diagnostic assay. Depending on context, the term “true negatives” may also refer to those subjects who do not have prostate cancer or a non-malignant disease of the prostate and who are categorized as such by the diagnostic assay.

Herein, the term “false positives” refers to those subjects who do not have a localized or a metastasised cancer of the prostate, a benign prostate disease, a precancerous prostatic lesion, or an acute or a chronic inflammation of prostatic tissue but are categorized by the diagnostic assay as having a localized or metastasised cancer of the prostate, a benign prostate disease, a precancerous prostatic lesion or an acute or chronic inflammation of prostatic tissue. Depending on context, the term “false positives” may also refer to those subjects who do not have prostate cancer or a non-malignant disease of the prostate but are categorized by the diagnostic assay as having prostate cancer or a non-malignant disease of the prostate.

The term “sensitivity”, as used herein in the context of its application to diagnostic assays, refers to the proportion of all subjects with localized or metastasised cancer of the prostate, a benign prostate disease, a precancerous prostatic lesion, or an acute or a chronic inflammation of prostatic tissue that are correctly identified as such (that is, the number of true positives divided by the sum of the number of true positives and false negatives).

The term “specificity” of a diagnostic assay, as used herein in the context of its application to diagnostic assays, refers to the proportion of all subjects with neither localized or metastasised cancer of the prostate nor a benign prostate disease, a precancerous prostatic lesion, or an acute or a chronic inflammation of prostatic tissue that are correctly identified as such (that is, the number of true negatives divided by the sum of the number of true negatives and false positives).

The term “adsorbent” refers to any material that is capable of accumulating (binding) a given biomolecule. The adsorbent typically coats a biologically active surface and is composed of a single material or a plurality of different materials that are capable of binding a biomolecule. Such materials include, but are not limited to, anion exchange materials, cation exchange materials, metal chelators, polynucleotides, oligonucleotides, peptides, antibodies, naturally occurring compounds, synthetic compounds, etc.

The phrase “biologically active surface” refers to any two- or three-dimensional extensions of a material that biomolecules can bind to, or interact with, due to the specific biochemical properties of this material and those of the biomolecules. Such biochemical properties include, but are not limited to, ionic character (charge), hydrophobicity, or hydrophilicity.

The phrase “binding biomolecule” refers to a molecule that displays an affinity for another biomolecule.

The term “immunogen” may be used interchangeably with the phrase “immunising agent” and refers to any substance or organism that provokes an immune response when introduced into the body of a given subject. All immunogens are considered as antigens and, in the context of the invention, can be defined on the basis of their immunogenicity, wherein “immunogenicity” refers to the ability of the immunogen to induce either a humoral or a cell-mediated immune response. In the context of the invention an immunogen that induces a “humoral immune response” activates antibody production and secretion by cells of the B-lymphocyte lineage (B-cells) and thus can be used to for antibody production as described herein. Such immunogens may be polysaccharides, proteins, lipids or nucleic acids, or they may be lipids or nucleic acids that are complexed to either a polysaccharide or a protein.

The term “solution” refers to a homogeneous mixture of two or more substances. Solutions may include, but are not limited to buffers, substrate solutions, elution solutions, wash solutions, detection solutions, standardisation solutions, chemical solutions, solvents, etc.

The phrase “coupling buffer” refers to a solution that is used to promote covalent binding of biomolecules to a biological surface.

The phrase “blocking buffer” refers to a solution that is used to (prevent) block unbound binding sites of a given biological surface from interacting with biomolecules in an unspecific manner.

The term “chromatography” refers to any method of separating biomolecules within a given sample such that the original native state of a given biomolecule is retained. Separation of a biomolecule from other biomolecules within a given sample for the purpose of enrichment, purification and/or analysis, may be achieved by methods including, but not limited to, size exclusion chromatography, ion exchange chromatography, hydrophobic and hydrophilic interaction chromatography, metal affinity chromatography, wherein “metal” refers to metal ions (e.g. nickel, copper, gallium, zinc, iron or cobalt) of all chemically possible valences, or ligand affinity chromatography wherein “ligand” refers to binding molecules, preferably proteins, antibodies, or DNA. Generally, chromatography uses biologically active surfaces as adsorbents to selectively accumulate certain biomolecules.

The phrase “mass spectrometry” refers to a method comprising employing an ionisation source to generate gas phase ions from a biological entity of a sample presented on a biologically active surface, and detecting the gas phase ions with an ion detector. Comparison of the time the gas phase ions take to reach the ion detector from the moment of ionisation with a calibration equation derived from at least one molecule of known mass allows the calculation of the estimated mass to charge ratio of the ion being detected.

The phrases “mass to charge ratio”, “m/z ratio” or “m/z” can be used interchangeably and refer to the ratio of the molecular weight (grams per mole) of an ion detected by mass spectrometry to the number of charges the ion carries. Thus a single biomolecule can be assigned more than one mass to charge ratio by a mass spectrometer if that biomolecule can be ionised into more than one species each of which carries a different number of charges.

The acronym “TOF” refers to the “time-of-flight” of a biomolecule or other molecular entity, such as an ion in a time-of-flight type mass spectrometer. “TOF” values are derived by measuring the duration of flight of an ion, typically between its entry into and exit from a time-of-flight analyser tube. Alternatively, the accuracy of TOF values can be improved by known methods, for example through the use of reflectrons and/or pulsed-laser ionization. TOF values for a given ion can be applied to previously established calibration equations derived from the TOF values for ions of known mass in order to calculate the mass to charge ratio of these ions.

The phrase “calibration equation” refers to a standard curve based on the TOF of biomolecules with known molecular mass. Application of a calibration equation to peaks in a mass spectrum allows the calculation of the m/z ratio of these peaks based on their observed TOF.

The phrase “laser desorption mass spectrometry” refers to a method comprising the use of a laser as an ionisation source to generate gas phase ions from a biomolecule presented on a biologically active surface, and detecting the gas phase ions with a mass spectrometer.

The term “mass spectrometer” refers to a gas phase ion spectrometer that includes an inlet system, an ionisation source, an ion optic assembly, a mass analyser, and a detector.

Within the context of the invention, the terms “detect”, “detection” or “detecting” refer to the identification of the presence, absence, or quantity of a given biomolecule.

The phrase “Mann-Whitney Rank Sum Test” refers to a non-parametric statistical method used to test the null hypothesis that two sets of values that do not have normal distributions are derived from the same population.

The phrase “energy absorbing molecule” and its acronym “EAM” refers to a molecule that absorbs energy from an energy source in a mass spectrometer thereby enabling desorption of a biomolecule from a biologically active surface. Cinnamic acid derivatives, sinapinic acid and dihydroxybenzoic acid, ferulic acid and caffeic acid are frequently used as energy-absorbing molecules in laser desorption of biomolecules. See U.S. Pat. No. 5,719,060 (Hutchens & Yip) for a further description of energy absorbing molecules.

The terms “peak” and “signal” may be used interchangeably, and refer to a defined, non-background value which is generated by a population of a given biomolecule of a certain molecular mass that has been ionised contacting the detector of a mass spectrometer, wherein the size of the population can be roughly related to the degree of the intensity of the signal. Typically, this “signal” can be defined by two values: an apparent mass-over-charge ratio (m/z) and an intensity value generated as described.

The phrases “peak intensity”, “intensity of a peak” and “intensity” may be used interchangeably, and refer to the relative amount of a biomolecule contacting the detector of a mass spectrometer in relation to other peaks in the same mass profile. Typically, the intensity of a peak is expressed as the maximum observed signal within a defined mass range that adequately defines the peak.

The phrases “signal to noise ratio”, “SN ratio” and “SN” may be used interchangeably, and refer to the ratio of a peak's intensity and a dynamically calculated value representing the average background signal detected in the approximate mass range of the peak. The SN ratio of a peak is typically used as an objective criterion for (a) computer-assisted peak detection and/or (b) manual evaluation of a peak as being an artefact.

The term “cluster” refers to a peak that is present in a certain set of mass spectra or mass profiles obtained from different samples belonging to two or more different groups (e.g. subjects with prostate cancer and healthy subjects). Within the set of spectra, the peaks or signals belonging to a given cluster can differ in their intensities, but not in the apparent molecular masses.

The term “classifier” refers to an algorithm or methodology which is using one or more defined traits or attributes to subdivide a population individual patients or samples or elements of data into a finite number of groups with as great a degree of accuracy as possible.

The term “tree” refers to a type of classifier consisting of a branching series of decision points (typically referred to as “leaves” or “nodes”) that eventually lead to the classification of individual patients or samples or elements of data from a population into one of a finite number of groups.

The phrase “mass profile” refers to a series of discrete, non-background noise peaks that are defined by their mass to charge ratio and are characteristic of an individual mass spectrum.

The acronym “ROC-AUC” refers to the area under a receiver operator characteristic curve. This is a widely accepted measure of diagnostic utility of some tool, taking into account both the sensitivity and specificity of the tool. Typically, ROC-AUC ranges from 0.5 to 1.0, where a value of 0.5 indicates the tool has no diagnostic value and a value of 1.0 indicates the tool has 100% sensitivity and 100% specificity.

The term “sensitivity” refers to the proportion of patients with the outcome in whom the results of the decision rule are abnormal. Typically, the outcome is disadvantageous to the patient. The term “specificity” refers to the proportion of patients without the outcome in whom the results of the decision rule are normal.

It is to be understood that the present invention is not limited to the particular materials and methods described or equipment, as these may vary. It is also to be understood that the terminology used herein is for the purpose of describing particular embodiments only, and is not intended to limit the scope of the present invention, which will be limited only by the appended claims.

It should be noted that as used herein and in the appended claims, the singular forms “a,” “an,” and “the” include plural reference unless the context clearly dictates otherwise. Thus, for example, a reference to “an antibody” is a reference to one or more antibodies and derivatives thereof known to those skilled in the art, and so forth.

### PSP94

PSP94 is a versatile protein that plays are role in several biological processes within the reproductive tract ranging from modulating the circulation of follicle-stimulating hormone (FSH) to inducing apoptosis in prostate cancer cells (Sheth et al. 1984; Chao et al. 1996; Hirano et al. 1996; Garde et al. 1999; Shukeir et al. 2003). It is one of the three major proteins secreted by the normal human prostate gland. As a secreted protein, this molecule is found in a variety of bodily fluids including serum (Teni et al. 1988; Reeves et al. 2005; van Huizen et al. 2005), urine (Teni et al. 1988; Liu et al. 1993), seminal plasma fluid (Sheth et al 1984; Dubé et al. 1987a; von der Kammer et al. 1991) and mucous gland secretions (Weiber et al. 1990). PSP94 occurs in both the free and bound forms in serum (Wu et al 1999).

Several groups have demonstrated that PSP94 has the clinical potential to becoming a relevant biomarker for prostate cancer (Dubé et al. 1987b; Tremblay et al. 1987; Abrahamsson et al. 1988; Teni et al. 1988; Abrahamsson et al. 1989; Teni et al. 1989; von der Kammer et al. 1990; Huang et al. 1993; Hyakutake et al. 1993; von der Kammer et al. 1993, Maeda et al. 1994; Tsurusaki et al. 1998, Sakai et al. 1999). Abnormal protein levels in serum are indicative of prostate cancer, wherein the irregular or erratic control of PSP94 secretion from the prostate is correlated with neoplasia (Wu et al. 1999). While most diagnostic methods utilising PSP94 as a discriminator for prostate cancer focus on detecting abnormal levels of the protein in serum samples (von der Kammer et al 1990; von der Kammer et al. 1993; Wu et al. 1999; U.S. Pat. No. 6,107,103; US 2006/0029984; WO 02/46448; WO 03/093474), others base their capabilities on detecting abnormal levels of PSP94 in urine samples (Teni et al. 1988; Teni et al. 1989) or in seminal plasma fluid (von der Kammer et al. 1990).

Full-length PSP94 has the following sequence:

(SEQ ID NO: 1; Accession No. AB29732.1/GI: 460569)
MNVLLGSVVIFATFVTLCNASCYFIPNEGVPGDSTRKCMDLKGNKHPINS
EWQTDNCETCTCYETEISCCTLVSTPVGYDKDNCQRIFKKEDCKYIVVEK
KDPKKTCSVSEWII

Mature PSP94 has the following sequence:

(SEQ ID NO: 2)
SCYFIPNEGVPGDSTRKCMDLKGNKHPINSEWQTDNCETCTCYETEISCC
TLVSTPVGYDKDNCQRIFKKEDCKYIVVEKKDPKKTCSVSEWII

### Diagnostic Tools

Although PSP94 has been shown to be a useful discriminatory factor for diagnosis and/or prognosis of prostate cancer, diagnostic tools utilizing this protein are both invasive and lacking sensitivity. A diagnostic tool utilising PSP94 and F/T PSA, which may or may not be standardized to creatinine, has not yet been described. This improves the discriminatory value for prostate cancer over each of the markers when used alone. Furthermore standardization of PSP94 to creatinine levels may also be utilized. In addition to this, urine samples are the preferred samples for diagnostic tools described herein, making the test ideal for clinical application. Embodiments of the invention are non-invasive and cost-effective.

The present invention relates to methods for differential diagnosis of prostate cancer or a non-malignant disease of the prostate by detecting PSP94 and F/T PSA, where PSP94 may be standardized to creatinine, within a biological sample of a given subject, comparing results with samples from healthy subjects, subjects having a non-malignant disease of the prostate and subjects having prostate cancer, wherein the comparison allows for the differential diagnosis of a subject as healthy, having non-malignant disease of the prostate or having prostate cancer.

One aspect of the invention includes a method for diagnosing prostate cancer in a subject comprising: (a) detecting a quantity, presence or absence of PSP94 and F/T PSA, where PSP94 may be standardized to creatinine, in a biological sample; and (b) classifying the subject as having or not having prostate cancer.

In an embodiment of the invention, the step of classifying the subject comprises comparing the quantity, presence or absence of PSP94 and F/T PSA, where PSP94 may be standardized to creatinine, with a reference value indicative of a prostate cancer. The reference value comprises PSP94 and F/T PSA, where PSP94 may be standardized to creatinine, previously characterised as being diagnostic for prostate cancer.

A further aspect of the invention includes a method for differential diagnosis of prostate cancer and non-malignant disease of the prostate in a subject, comprising: (a) detecting a quantity, presence or absence of PSP94 and F/T PSA, where PSP94 may be standardized to creatinine, in a biological sample; and (b) classifying the subject as having prostate cancer, non-malignant disease of the prostate, or as healthy, based on the quantity, presence or absence of PSP94 and F/T PSA, where PSP94 may be standardized to creatinine, in the biological sample.

In an embodiment of the invention, the step of classifying the subject comprises comparing the quantity, presence or absence of PSP94 and F/T PSA, where PSP94 may be standardized to creatinine, with a reference value indicative of prostate cancer and a reference value indicative of a non-malignant disease of the prostate. The reference values comprise PSP94 and F/T PSA, where PSP94 may be standardized to creatinine, characterised as being diagnostic for prostate cancer or for a non-malignant disease of the prostate.

A further aspect of the invention includes a method for differential diagnosis of healthy, non-malignant disease of the prostate, precancerous prostatic lesion, localized cancer of the prostate, metastasised cancer of the prostate, and acute or chronic inflammation of prostatic tissue in a subject, comprising: (a) detecting the quantity, presence or absence of PSP94 and F/T PSA, where PSP94 may be standardized to creatinine, in a biological sample; and (b) classifying the subject as having non-malignant disease of the prostate, precancerous prostate lesion, localized cancer of the prostate, metastasised cancer of the prostate, and/or acute or chronic inflammation of prostatic tissue, or as healthy, based on the quantity, presence or absence of PSP94 and F/T PSA, where PSP94 may be standardized to creatinine, in the biological sample. Each of the reference values comprise values for good health, non-malignant disease of the prostate, precancerous prostate lesion, localized cancer of the prostate, metastasised cancer of the prostate, and/or acute or chronic inflammation of prostatic tissue.

In one embodiment of the invention, a method for differential diagnosis of prostate cancer or a non-malignant disease of the prostate comprises: contacting a biological sample with an adsorbent present on a biologically active surface under specific binding conditions, allowing the biomolecules within the biological sample to bind to said adsorbent, detecting one or more bound biomolecules using a detection method, wherein the detection method generates a mass profile of said sample, transforming the mass profile generated into a computer-readable form, and comparing the mass profile of said sample with a database containing mass profiles from comparable samples specific for healthy subjects, subjects having prostate cancer, and/or subjects having a non-malignant disease of the prostate. An outcome of said comparison will allow for the determination of whether the subject from which the biological sample was obtained, is healthy, has a non-malignant disease of the prostate and/or prostate cancer based on the presence, absence or comparative quantity of specific biomolecules.

In one embodiment, a biologically active surface comprises an adsorbent comprising silicon dioxide molecules. In another embodiment, a biologically active surface comprises an adsorbent comprised of antibodies. Antibodies may be antibodies specific to PSP94 and PSA. Biologically active surfaces useful for practicing the methods of the invention are further described in greater detail below.

A quantity, presence, or absence of PSP94 and F/T PSA, where PSP94 may be standardized to creatinine, in a biological sample obtained from a subject may be determined by mass spectrometry. A method of mass spectrometry may be selected from the group consisting of matrix-assisted laser desorption time/time of flight (MALDI-TOF), surface enhanced laser desorption ionisation/time of flight (SELDI-TOF), liquid chromatography, MS-MS, or ESI-MS. Detection methods useful for practicing the methods of the invention are further described in greater detail below.

In addition, other methods of determining a quantity, presence or absence of PSP94 and F/T PSA, where PSP94 may be standardized to creatinine, in a biological sample can be utilized, such as ELISA utilizing antibodies targeted to a biomarker of the invention. In any of the embodiments of the methods described above, PSP94 and F/T PSA, where PSP94 may be standardized to creatinine, may be detected within a given biological sample. Detection of biomolecules of the invention is based on specific sample pre-treatment conditions, the pH of binding conditions, the adsorbent used on the biologically active surface, and the calibration equation used to determine the TOF of the given biomolecules.

In one embodiment of the invention, a biomolecule of the invention can include PSP94 and F/T PSA, where PSP94 may be standardized to creatinine, and may be used individually to diagnose a subject as being healthy, or having a non-malignant disease of the prostate, or having a precancerous prostatic lesion, or having a localized cancer of the prostate, or having a metastasised cancer of the prostate, or having an acute or a chronic inflammation of prostatic tissue. In another embodiment of the invention, biomolecules that can include PSP94 and F/T PSA, where PSP94 may be standardized to creatinine, may be used in combination with one another to diagnose a subject as being healthy, or having of a non-malignant disease of the prostate, or having a precancerous prostatic lesion, or having a localized cancer of the prostate, or having a metastasised cancer of the prostate, or having an acute or a chronic inflammation of prostatic tissue.

In yet another embodiment of the invention, detection and/or quantification of biomolecules, including PSP94 and F/T PSA, where PSP94 may be standardized to creatinine, may be used in combination with another diagnostic tool to diagnose a subject as being healthy, or having a non-malignant disease of the prostate, or having a precancerous prostatic lesion, or having a localized cancer of the prostate, or having a metastasised cancer of the prostate, or having an acute or a chronic inflammation of prostatic tissue. For example, PSP94 and F/T PSA, where PSP94 may be standardized to creatinine, may be used in combination with other diagnostic tools specific for prostate cancer detection such as, but not limited to, DRE, rectal palpitation, biopsy evaluation using Gleason scoring, radiography and symptomological evaluation by a qualified clinician.

Methods for detecting biomolecules according to the invention have many applications. For example, PSP94 and FIT PSA, where PSP94 may be standardized to creatinine, can be measured to differentiate between healthy subjects, subjects having a non-malignant disease of the prostate, subjects having a precancerous prostatic lesion, or subjects having a localized cancer of the prostate, or subjects having a metastasised cancer of the prostate, or subjects with an acute or a chronic inflammation of prostatic tissue, and thus are useful as an aid in diagnosis of a non-malignant disease of the prostate, or a precancerous prostatic lesion, or a localized cancer of the prostate, or a metastasised cancer of the prostate, or an acute or a chronic inflammation of prostatic tissue. Alternatively, said biomolecules may be used to diagnose a subject as being healthy.

Another aspect of the invention includes an in vitro method for diagnosis of a prostate cancer in a subject comprising detecting differentially expressed biomarkers in a biological sample by: (a) contacting a sample with a binding molecule specific for PSP94 and PSA, and (b) detecting a quantity, presence or absence of PSP94 and F/T PSA, where PSP94 may be standardized to creatinine, wherein the quantity, presence or absence of PSP94 and F/T PSA, where PSP94 may be standardized to creatinine, allows for diagnosis of the subject as healthy or having prostate cancer.

A further aspect of the invention includes a method for in vitro differential diagnosis of prostate cancer and non-malignant disease of the prostate in a subject, comprising detecting one or more differentially expressed biomarkers in a biological sample: (a) contacting a sample with a binding molecule specific for PSP94 and PSA, and (b) detecting a quantity, presence or absence of PSP94 and F/T PSA, where PSP94 may be standardized to creatinine, in the sample, wherein the quantity, presence or absence of PSP94 and F/T PSA, where PSP94 may be standardized to creatinine, where biomarker(s) allows for differential diagnosis of the subject as having prostate cancer, and/or having a non-malignant disease of the prostate, or as being healthy.

Still a further aspect of the invention includes an in vitro method for differential diagnosis of healthy, prostate cancer, non-malignant disease of the prostate, precancerous prostatic lesion, localized cancer of the prostate, metastasised cancer of the prostate, and acute or chronic inflammation of prostatic tissue in a subject, comprising detection of PSP94 and F/T PSA, where PSP94 may be standardized to creatinine, in a biological sample by: (a) contacting a sample with a binding molecule specific for PSP94 and PSA, and (b) detecting a quantity, presence or absence of PSP94 and F/T PSA, where PSP94 may be standardized to creatinine, wherein the presence or absence of PSP94 and F/T PSA, where PSP94 may be standardized to creatinine, allows for the differential diagnosis of the subject as healthy, having non-malignant disease of the prostate, precancerous prostate lesions, localized cancer of the prostate, metastasised cancer of the prostate, and/or having acute or chronic inflammation of the prostate, or as being healthy.

An in vitro binding assay can be used to detect PSP94 and F/T PSA, where PSP94 may be standardized to creatinine, within a biological sample of a given subject. A given biomolecule of the invention can be detected within a biological sample by contacting the biological sample from a given subject with specific binding molecule(s) under conditions conducive for an interaction between the given binding molecule(s) and a biomolecule that can be PSP94 and PSA.

If a given biomolecule is present in a biological sample, it will form a complex with its binding molecule. To determine if a quantity of the detected biomolecule in a biological sample is comparable to a given quantity for healthy subjects, subjects having a non-malignant disease of the prostate, subjects having a precancerous prostatic lesion, subjects having a localized cancer of the prostate, subjects having a metastasised cancer of the prostate or subjects with an acute or a chronic inflammation of prostatic tissue, the amount of the complex formed between a binding molecule and a biomolecule, which can be PSP94 and PSA, can be determined by comparing to a standard. For example, if the amount of the complex falls within a quantitative value for healthy subjects, then the sample can be considered to be obtained from a healthy subject. If the amount of the complex falls within a quantitative value for subjects known to have a non-malignant disease of the prostate, then the sample can be considered to be obtained from a subject having a non-malignant disease of the prostate. If the amount of the complex falls within a quantitative range for subjects known to have prostate cancer, then the sample can be considered to have been obtained from a subject having prostate cancer. In vitro binding assays that are included within the scope of the invention are those known to the skilled in the art (i.e. ELISA, western blotting).

In further aspects, an embodiment of the invention further provides in vitro methods for differential diagnosis of prostate cancer or a non-malignant disease of the prostate comprising: detecting of one or more differentially expressed biomolecules that can include PSP94 and F/T PSA, where PSP94 may be standardized to creatinine, within a given biological sample. This method comprises obtaining a biological sample from a subject, contacting said sample with a binding molecule specific for a differentially expressed biomolecule, detecting an interaction between the binding molecule and its specific biomolecule, wherein the detection of an interaction indicates the presence or absence of said biomolecule, thereby allowing for the differential diagnosis of a subject as healthy, or having a non-malignant disease of the prostate, or having a precancerous prostatic lesion, or having a localized cancer of the prostate, or having a metastasised cancer of the prostate, or having an acute or a chronic inflammation of prostatic tissue.

Binding molecules include, but are not limited to, nucleic acids, nucleotides, polynucleotides, amino acids, polypeptides (e.g., monoclonal and/or polyclonal antibodies, antigens, etc.), carbohydrates (e.g., sugars), fatty acids, lipids, steroids, or combinations thereof (e.g. glycoproteins, ribonucleoproteins, lipoproteins), compounds or synthetic molecules. In one preferred embodiment, binding molecules are antibodies specific for PSP94 or PSA. Biomolecules detected using the above-mentioned binding molecules include, but are not limited to, molecules comprising nucleic acids, nucleotides, polynucleotides, amino acids, polypeptides, (e.g., monoclonal and/or polyclonal antibodies, antigens, etc.) carbohydrates (e.g., sugars), fatty acids, lipids, steroids, and combinations thereof (e.g., glycoproteins, ribonucleoproteins, lipoproteins).

For example, antibodies or fragments thereof may be utilised for the detection of PSP94 and PSA, in a biological sample comprising: applying a labelled antibody directed against a given biomolecule of the invention to said biological sample under conditions that favour an interaction between the labelled antibody and its corresponding biomolecule. Depending on the nature of a biological sample, it is possible to determine not only presence of a biomolecule, but also its cellular distribution. For example, in a blood serum sample, only serum levels of a given biomolecule can be detected, whereas its level of expression and cellular localisation can be detected in histological samples.

In another example, an antibody directed against a biomolecule of the invention that is coupled to an enzyme is detected using a chromogenic substrate that is recognised and cleaved by the enzyme to produce a chemical moiety, which is readily detected using spectrometric, fluorimetric or visual means. Enzymes used to for labelling include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Detection may also be accomplished by visual comparison of the extent of the enzymatic reaction of a substrate with that of similarly prepared standards. Alternatively, radio-labelled antibodies can be detected using a gamma or a scintillation counter, or they can be detected using autoradiography. In another example, fluorescently labelled antibodies are detected based on the level at which the attached compound fluoresces following exposure to a given wavelength. Fluorescent compounds typically used in antibody labelling include, but are not limited to, fluorescein isothiocynate, rhodamine, phycoerthyrin, phycocyanin, allophycocyani, o-phthaldehyde and fluorescamine. In yet another example, antibodies coupled to a chemi- or bioluminescent compound can be detected by determining the presence of luminescence. Such compounds include, but are not limited to, luminal, isoluminal, theromatic acridinium ester, imidazole, acridinium salt, oxalate ester, luciferin, luciferase and aequorin.

Furthermore, in vivo techniques for detecting a biomolecule include introducing into a subject a labelled antibody directed against a biomolecule, which can be PSP94 and PSA.

In addition, methods of the invention for differential diagnosis of healthy subjects, subjects having a non-malignant disease of the prostate, subjects having a precancerous prostatic lesion, subjects having a localized cancer of the prostate, subjects having a metastasised cancer of the prostate and/or subjects having an acute or chronic inflammation of prostatic tissue, described herein may be combined with other diagnostic methods to improve the outcome of the differential diagnosis.

Methods of the invention can also be used for differential diagnosis of healthy subjects, subjects having a precancerous prostatic lesions, subjects having a non-malignant disease of the prostate, subjects having a localized cancer of the prostate, subjects having metastasised cancer of the prostate, and/or subjects having acute or chronic inflammation of the prostate, or any two or more of the above states.

In general, for an equivalent number of patients categorized (i.e., for a data set of the same size), one would expect a database divided into three classes (healthy, having non-malignant disease of the prostate, having prostate cancer) to have a greater diagnostic accuracy when used for diagnosing patients, as compared to a database divided into six classes (healthy, having non-malignant disease of the prostate, having localized cancer of the prostate, having metastasised cancer of the prostate, having precancerous prostatic lesions, and having acute or chronic inflammation of prostatic tissue). One would also reasonably expect that an increase in the data characterized (i.e., number of patients entered into the database) would result in an improvement in the diagnostic accuracy of the database. The invention can also be used for the differential diagnosis of any two or more of the six classes described herein.

### Biological Samples of the Invention

Typically, PSP94 and PSA are detected in urine samples, but their detection is not limited to urine samples. Biomolecules of the invention can be detected in blood, blood serum, blood plasma, urine, semen, seminal fluid, seminal plasma, prostatic fluid, pre-ejaculatory fluid (Cowper's fluid), excreta, tears, saliva, sweat, biopsy, ascites, cerebrospinal fluid, lymph, or tissue extract (biopsy) samples. Preferably, biological samples used to detect biomolecules of the invention are urine, semen, seminal fluid, seminal plasma, prostatic fluid, pre-ejaculatory fluid (Cowper's fluid).

Furthermore, biological samples can be isolated from mammalian subjects, preferably humans.

A subject that is said to have a prostate cancer possesses morphological, biochemical and functional alterations of their prostatic tissue such that the tissue can be characterised as a malignant neoplasm. The stage to which a prostate cancer has progressed can be determined using known methods currently available to those skilled in the art [e.g. Union Internationale Contre Cancer (UICC) system or American Joint Committee on Cancer (AJC)]. Currently, the most widely used method for determining the extent of malignancy of a prostatic neoplasm is the Gleason Grading system. Gleason grading is based exclusively on the architectural pattern of the glands of a prostatic neoplasm, wherein the ability of neoplastic cells to structure themselves into glands resembling those of the normal prostate is evaluated using a scale of 1 to 5. For example, neoplastic cells that are able to architecturally structure themselves such that they resemble normal prostate gland structure are graded 1-2, whereas neoplastic cells that are unable to do so are graded 4-5. As known to those skilled in the art, a prostatic neoplasm whose tumour structure is nearly normal will tend to behave, biologically, as normal tissue and therefore it is unlikely that it will be aggressively malignant. Gleason score may be integrated with other grading methods and/or staging systems to determine cancer stage.

A subject is said to have a non-malignant disease of the prostate possesses morphological and/or biochemical alterations of their prostatic tissue but does not exhibit malignant neoplastic properties known to those skilled in the art. Such diseases include, but are not limited to, inflammatory and proliferative lesions, as well as benign disorders of the prostate. Within the context of the invention, inflammatory lesions encompass acute and chronic bacterial prostatitis, as well as chronic abacterial prostatitis, proliferative lesions include benign prostate hyperplasia (BPH).

### Biologically Active Surfaces

Biologically active surfaces include, but are not limited to, surfaces that contain adsorbents with anion exchange properties (adsorbents that are positively charged), cation exchange properties (adsorbents that are negatively charged), hydrophobic properties, reverse phase chemistry, groups such as nitriloacetic acid that immobilize metal ions such as nickel, gallium, copper, or zinc (metal affinity interaction), or biomolecules such as proteins, antibodies, nucleic acids, or protein binding sequences, covalently bound to the surface via carbonyl diimidazole moieties or epoxy groups (specific affinity interaction).

These surfaces may be located on matrices like polysaccharides such as sepharose, e.g. anion exchange surfaces or hydrophobic interaction surfaces, or solid metals, e.g. antibodies coupled to magnetic beads or a metal surface. Surfaces may also include gold-plated surfaces such as those used for Biacore Sensor Chip technology.

Biologically active surfaces are able to adsorb biomolecules like nucleotides, nucleic acids, polynucleotides, amino acids, polypeptides (e.g., monoclonal and/or polyclonal antibodies), steroids, carbohydrates (e.g., sugars), fatty acids, lipids, hormones, and combinations thereof (e.g., glycoproteins, ribonucleoproteins, lipoproteins).

Devices that use biologically active surfaces to selectively adsorb biomolecules can be chromatography columns for Fast Protein Liquid Chromatography (FPLC) and High Pressure Liquid Chromatography (HPLC), where the matrix, e.g. a polysaccharide, carrying the biologically active surface, is filled into vessels (usually referred to as “columns”) made of glass, steel, or synthetic materials like polyetheretherketone (PEEK).

In yet another embodiment, devices that use biologically active surfaces to selectively adsorb biomolecules may be metal strips carrying thin layers of a biologically active surface on one or more spots of the strip surface to be used as probes for gas phase ion spectrometry analysis, for example the PS20 ProteinChip array for (Ciphergen Biosystems, Inc.) for SELDI analysis.

### Detection of Biomolecules of the Invention

In one embodiment, mass spectrometry can be used to detect biomolecules, which can be PSP94 and PSA, of a given sample. Such methods include, but are not limited to, matrix-assisted laser desorption flight/time-of-flight (MALDI-TOF), surface-enhanced laser desorption flight/time-of-flight (SELDI-TOF), liquid chromatography coupled with MS, MS-MS, or ESI-MS. Typically, biomolecules are analysed by introducing a biologically active surface containing said biomolecules, ionising said biomolecules to generate ions that are collected and analysed.

In a preferred embodiment, PSP94 and/or PSA are detected in samples using gas phase ion spectrometry, and more preferably, using mass spectrometry. In one embodiment, matrix-assisted laser desorption/ionisation (“MALDI”) mass spectrometry can be used. MALDI is a well known technique and is described in Brummell et al., Science 264: 399-402 (1994), which is hereby incorporated by reference. In MALDI, a sample is partially purified to obtain a fraction that comprises a biomolecule by employing such separation methods as: two-dimensional gel electrophoresis (2D-gel) or high performance liquid chromatography (HPLC). Specifically, sample(s) and matrix with a positive charge are mixed together and flashed with a laser. The matrix becomes ionized (MH+) with an extra proton and then the proton is transferred to the sample to create a positively charged sample(s). The charged sample(s) is then run through a detector where the smaller ions reach the detector first and then the larger ions. This is the time of flight (TOF), and the mass to charge ratio (M/Z) is proportional to the square of the drift time.

In another embodiment, surface-enhanced laser desorption/ionisation mass spectrometry (SELDI) can be used to detect a biomolecule, which can be PSP94 and/or PSA, and uses a substrate comprising adsorbents to capture biomolecules, which can then be directly desorbed and ionised from the substrate surface during mass spectrometry. Since the substrate surface in SELDI captures biomolecules, a sample need not be partially purified as in MALDI. However, depending on the complexity of a sample and the type of adsorbents used, it may be desirable to prepare a sample to reduce its complexity prior to SELDI analysis. The SELDI is described, inter alia, in U.S. Pat. Nos. 5,719,060, 6,225,047, 6,579,719, and 6,818,411, which are hereby incorporated by reference.

In a preferred embodiment, a laser desorption time-of-flight mass spectrometer is used with a probe of the present invention. In laser desorption mass spectrometry, biomolecules bound to a biologically active surface are introduced into an inlet system. Biomolecules are desorbed and ionised into the gas phase by a laser. Generated ions are then collected by an ion optic assembly. These ions are accelerated through a short high-voltage field and allowed to drift into a high vacuum chamber of a time-of-flight mass analyser. At the far end of the high vacuum chamber, accelerated ions collide with a detector surface at varying times. Since the time-of-flight is a function of the mass of the ions, the elapsed time between ionisation and impact can be used to identify the presence or absence of molecules of a specific mass.

Data analysis can include determining signal strength (e.g., intensity of peaks) of a biomolecule(s) detected and removing “outliers” (data deviating from a predetermined statistical distribution). An example of this is the normalization of peaks, a process whereby the intensity of each peak relative to some reference is calculated. For example, a reference can be background noise generated by the instrument and/or chemicals (e.g., energy absorbing molecule), which is set as zero in the scale. Then the signal strength detected for each biomolecule can be displayed in the form of relative intensities in the scale desired (e.g., 100). Alternatively, the observed signal for a given peak can be expressed as the ratio of the intensity of that peak over the sum of the entire observed signal for both peaks and background noise in a specified mass to charge ratio range. Alternatively, a standard may be admitted with the sample so that a peak from the standard can be used as a reference to calculate relative intensities of the signals observed for each biomolecule(s) detected.

Resulting data can be transformed into various formats for display, typically through the use of computer algorithms. In one format, referred to as a “spectrum view”, a standard spectral view can be displayed, wherein the view depicts the quantity of a biomolecule reaching the detector at each possible mass to charge ratio. In another format, referred to as “scatter plot”, only the intensity and mass to charge information for defined peaks are retained from the spectrum view, yielding a cleaner image and enabling biomolecules with nearly identical molecular mass to be more easily distinguished from one another.

Using any of the above display formats, it can be readily determined from a signal display whether a biomolecule having a particular TOF is detected from a sample. Preferred biomolecules of the invention are PSP94 and PSA, for determining F/T PSA and where PSP94 can be standardized to creatinine.

In another aspect of the invention, biomolecules (e.g., PSP94 and F/T PSA) can be detected using other known methods. For example, an in vitro binding assay can be used to detect a biomolecule within a biological sample of a given subject. A given biomolecule can be detected within a biological sample by contacting the biological sample from a given subject with specific binding molecule(s) under conditions conducive for an interaction between the given binding molecule(s) and a biomolecule. Binding molecules include, but are not limited to, nucleic acids, nucleotides, polynucleotides, amino acids, polypeptides (e.g., monoclonal and/or polyclonal antibodies, and antigens), carbohydrates (e.g., sugars), fatty acids, lipids, steroids, or combinations thereof. (e.g. glycoproteins, ribonucleoproteins, lipoproteins), compounds or synthetic molecules. Preferably, binding molecules are antibodies specific for PSP94 or PSA. Biomolecules detected using the above-mentioned binding molecules include, but are not limited to, molecules comprising nucleic acids, nucleotides, polynucleotides, amino acids, polypeptides (e.g., monoclonal and/or polyclonal antibodies, antigens), carbohydrates (e.g., sugars), fatty acids, lipids, steroids, and combinations thereof (e.g., glycoproteins, ribonucleoproteins, lipoproteins).

### Sandwich Assay

Sandwich assays for detecting a biomolecule, which can be PSP94 and PSA can be used as a diagnostic tool for diagnosis of a subject as being healthy, having a non-malignant disease of the prostate, having a precancerous prostatic lesion, having a localized cancer of the prostate, or a metastasised cancer of the prostate, or having an acute or a chronic inflammation of prostatic tissue. In the context of the invention, sandwich assays comprise attaching a monoclonal antibody to a solid surface such as a plate, tube, bead, or particle, wherein the antibody is preferably attached to the well surface of a 96-well microtitre plate. A pre-determined volume of sample (e.g., serum, urine, tissue cytosol) containing a subject biomarker is added to the solid phase antibody, and the sample is incubated for a period of time at a pre-determined temperature conducive for specific binding of subject biomarkers within the given sample to the solid phase antibody. Following incubation, the sample fluid is discarded, and the solid phase is washed with buffer to remove any unbound material. A second monoclonal antibody (to a different determinant on the subject biomarker) is added to the solid phase. This antibody is labelled with a detector molecule or atom (e.g., enzyme, fluorophore, chromophore, or ¹²⁵I), and the solid phase is incubated with the second antibody. The second antibody is decanted and the solid phase is washed with buffer to remove unbound material.

The amount of bound label, which is proportional to the amount of subject biomarker present in the sample, is quantitated.

### Kits

A further aspect of the invention comprises a kit for diagnosing a prostate disease within a subject comprising: a biologically active surface comprising an adsorbent, binding solutions, and instructions to use the kit, wherein the instructions outline a method for diagnosis of a prostate cancer in a subject or a method for differential diagnosis of healthy, non-malignant disease of the prostate, precancerous prostatic lesion, localized cancer of the prostate, metastasised cancer of the prostate, and acute or chronic inflammation of prostatic tissue in a subject according to the invention.

Any of the biologically active surfaces described herein may be used to practice the invention. In an embodiment of the invention, a biologically active surface may comprise an adsorbent comprising of silicon dioxide molecules. In another embodiment of the invention, a biologically active surface may comprise an adsorbent comprising antibodies specific to PSP94 and F/T PSA.

A further aspect of the invention comprises a kit for diagnosing prostate disease within a subject comprising a binding solution, a binding molecule, a detection substrate, and instructions, wherein the instructions describe an in vitro method for diagnosis of a prostate cancer in a subject, an in vitro method for differential diagnosis of prostate cancer and non-malignant disease of the prostate in a subject, or an in vitro method for differential diagnosis of healthy, prostate cancer, non-malignant disease of the prostate, precancerous prostatic lesion, localized cancer of the prostate, metastasised cancer of the prostate, and acute or chronic inflammation of prostatic tissue in a subject.

Yet another aspect of the invention comprises kits using methods of the invention as described in another section for differential diagnosis of prostate cancer or a non-malignant disease of the prostate, wherein the kits are used to detect biomolecules, which can be PSP94 and F/T PSA, where PSP94 may be standardized to creatinine.

Methods used to detect biomolecules, which can be PSP94 and F/T PSA can also be used to determine whether a subject is at risk of developing prostate cancer or has developed prostate cancer. Such methods may also be employed in the form of a diagnostic kit comprising a binding molecule specific to a biomolecule, which can be PSP94 and PSA, solutions and materials necessary for the detection of a biomolecule of the invention, and instructions to use the kit based on the above-mentioned methods.

For example, a kit can be used to detect biomolecules such as PSP94 and PSA and have many applications. For example, kits can be used to differentiate whether a subject is healthy, has a non-malignant disease of the prostate, or a prostate cancer, thus aiding diagnosis of a prostate cancer and/or a non-malignant disease of the prostate. Moreover, kits can be used to differentiate whether a subject is healthy, having a non-malignant disease of the prostate, has a precancerous prostatic lesion, has a localized cancer of the prostate, has a metastasised cancer of the prostate, or has an acute or a chronic inflammation of the prostate.

In an embodiment, a kit may comprise instructions on how to use the kit, a biologically active surface comprising an adsorbent, wherein the adsorbent is suitable for binding one or more biomolecules of the invention, a denaturation solution for the pre-treatment of a sample, a binding solution, and one or more washing solution(s) or instructions for making a denaturation solution, binding solution, or washing solution(s), wherein the combination of solutions allows for the detection of a biomolecule using gas phase ion spectrometry. Such kits can be prepared from materials described in other previously detailed sections (e.g., denaturation buffer, binding buffer, adsorbents, washing solution(s), etc.).

In another embodiment, a kit may comprise a first substrate comprising an adsorbent thereon (e.g., a particle functionalised with an adsorbent) and a second substrate onto which the first substrate can be positioned to form a probe, which is removably insertable into a gas phase ion spectrometer. In other embodiments, a kit may comprise a single substrate, which is in the form of a removably insertable probe with adsorbents on the substrate.

In another embodiment, a kit may comprise a binding molecule(s) that specifically binds to a biomolecule, which can be PSP94 and/or PSA, a detection reagent, appropriate solutions and instructions on how to use the kit. Such kits can be prepared from materials described above and known materials. A binding molecule used within such a kit may include, but is not limited to, nucleic acids, nucleotides, polynucleotides, amino acids, polypeptides (e.g., monoclonal and/or polyclonal antibodies), carbohydrates (e.g., sugars), fatty acids, lipids, steroids, hormones, or a combination thereof (e.g. glycoproteins, ribonucleoproteins, lipoproteins), compounds or synthetic molecules. In another embodiment, a kit comprises a binding molecule or panel of binding molecules that specifically bind to PSP94 and/or PSA, a detection reagent, appropriate solutions and instructions on how to use the kit. Each binding molecule would be distinguishable from every other binding molecule in a panel of binding molecules, yielding easily interpreted signal for each of the biomolecules detected by the kit. Such kits can be prepared from the materials described above and known materials. A binding molecule can include, but is not limited to, nucleic acids, nucleotides, polynucleotides, amino acids, polypeptides (e.g., monoclonal and/or polyclonal antibodies), carbohydrates (e.g., sugars), fatty acids, lipids, steroids, hormones, or a combination thereof (e.g. glycoproteins, ribonucleoproteins, lipoproteins), compounds or synthetic molecules.

In any of the embodiments described above, a kit may optionally further comprise a standard or control biomolecule so that the biomolecules detected within the biological sample can be compared with said standard to determine if the test amount of a marker detected in a sample is a diagnostic amount consistent with a diagnosis of a non-malignant disease of the prostate, a precancerous prostatic lesion, localized cancer of the prostate, metastasised cancer of the prostate, acute or a chronic inflammation of the prostate. Likewise, a biological sample can be compared with said standard to determine if the test amount of a marker detected is said sample is a diagnostic amount consistent with a diagnosis as healthy.

Patients with hypertension can have higher PSP94 values, even in the absence of non-malignant prostate disease or prostate cancer. In any of the embodiments described above, a method of diagnosis includes methods where a subject with hypertension is excluded from said method. Additionally, any database entries of the described embodiments and/or reference values can be obtained from a population of subjects, wherein the population of subjects excludes subjects with hypertension.

The present invention is further illustrated by the following examples, which should not be construed as limiting in any way.

### EXAMPLES

### Example 1

### Samples Used for Biomarker Discovery

Patients were recruited through a series of urological clinics and hospitals located in southern British Columbia (2 sites), Quebec (1 site), Manitoba (1 site), Nova Scotia (1 site) and Ontario (15 sites) for a pre-biopsy screening evaluation. Spot urine samples were collected without a preceding DRE. 24-hour urine samples were obtained prospectively no more than ten days prior to the patient undergoing a previously scheduled biopsy of the prostate for suspicion of prostate cancer. Serum samples were obtained by standard blood draw and collected as a 10 mL sample volume.

Patients were recruited for sample collection for the Pre-Biopsy Screening provided they were able to meet the following criteria:
Patient was male, at least 50 years of age and able to understand, and is willing to sign a written informed consent document.
Patient was previously scheduled for a biopsy of the prostate for suspicion of prostate cancer.
Patient could provide urine samples for analysis and serum samples for total PSA testing.
Patient had complete medical history information available (including tumour stage and grade if the patient was subsequently diagnosed as having prostate cancer).
   Patients were excluded when:
Patient reported a previous incidence of prostate cancer.
Patient reported a previous incidence of non-prostate cancer except basal skin cell carcinoma in the previous two years.
Patient reported taking either investigational agents or any prescribed pre-operative medications at the time of sample collection.

### Patient Clinical/Medical History Information

Medical history information was obtained as close to the time of sample collection as possible. This information included: age of patient; circulating PSA levels at time of sample collection; pathology and history of prostate cancer; presence of other chronic or acute conditions unrelated to prostate cancer at the time of sample collection and current management as well as current and past treatment regimes for prostate cancer.

### Sample Groups

Aggressive prostate cancer is defined as Gleason score of ≧7 or non-cancer/non-aggressive cancer (Gleason score ≦6). Non-cancer samples included:
Patients diagnosed with a non-malignant disease of the prostate (for example, benign prostatic hyperplasia). Confirmation of the absence of prostate cancer was evaluated by histological examination of prostatic tissue (needle point biopsy).
Prostatic intraepithelial neoplasia (PIN) samples: patients were diagnosed as having the disease by confirmation of the presence of PIN through post-surgical histological evaluation (biopsy).
Non-PCa/PIN samples: patients were diagnosed as being free of disease by confirmation of the absence of prostate cancer/PIN as evaluated by histological examination of prostatic tissue (biopsy).
Control samples: patients with no reported complaints or symptoms related to prostate cancer, and who were not suffering from severe disease at the time of collection.

TABLE 1
Patient Samples test for PSP94 and F/T PSA that had
Total PSA values from 2.5 to 10 ng/mL

| | Aggressive | | | |
|---|---|---|---|---|
| | Prostate | Gleason | Non | |
| | Cancer | score 6 | Cancer | Total |
| Total | 18 | 26 | 44 | 88 |
| Samples in | | | | |
| Study | | | | |
| Men without | 7 | 15 | 22 | 44 |
| hypertension | | | | |

### Sample Handling

Samples originating from sites outside of Winnipeg, Manitoba were shipped frozen on dry ice. Those samples obtained from Victoria General Hospital (Winnipeg, Manitoba) were frozen at the site and then transported on dry ice to the laboratory. Those samples obtained from the Winnipeg Clinic were stored at 4° C. at the site for same-day pickup. These samples were then transported on ice to the laboratory. Upon receipt urine samples stored at −20° C.

### Example 2

### Immunoassay of Urine PSP94 in a Microsphere Multiplex System

### Microsphere Preparation:

Microspheres were coated with 10 μg polyclonal anti-PSP94 from R&D Systems antibody per 1,250,000 beads using the BioRad coupling procedure for the Amine Coupling Kit.

### Preparation of Calibrators and Controls:

PSP94 obtained from R&D Systems was diluted in Assay Buffer to a create a 1000 ng/mL stock solution. Dilutions were made from the 1000 ng/mL stock to create calibrators and controls that ranged from 0.5 ng/mL to 12 ng/mL.

Sample Preparation before Testing:

Aliquoted urine samples were stored at −20° C. On the day of immunoassay testing, the thawed samples were centrifuged for 4 minutes at 13,000 RPM (16,000×g) on a Heraeus® Biofuge. The samples were diluted to 1:20 in a PBS Buffer containing bovine serum albumin (Assay buffer).

### Immunoassay to Detection of PSP94 in Urine:

This assay uses a quantitative sandwich enzyme immunoassay format.
1. The polyclonal antibody specific for PSP94 coupled onto microspheres are vortexed and sonicated. Fifty microliters (50 μL) of resuspended microspheres are then pipetted into a Millipore® microfilter plate and washed with PBS and 0.05% Tween® buffer (Wash Buffer).
2. The washed beads are followed by the addition of 50 μL of either PSP94 calibrators (0 to 12 ng/mL), controls (2 or 6 ng/ml) or urine samples diluted into Assay Buffer. PSP94 present in urine binds to the polyclonal antibody attached to the microspheres.
3. After washing away unbound substances with Wash Buffer, 1:400 dilution of a mouse anti-PSP94 monoclonal antibody (Novus Biologicals) in Assay Buffer is added to each well.
4. After washing away unbound substances with Wash Buffer a 1:100 dilution of a goat anti-mouse antibody linked to phycoerythrin (Jackson Immunoresearch) in Assay Buffer is added to each well. The wells are washed with Wash Buffer and resuspended in 130 uL of Assay Buffer.
5. The filter plates are placed in the BioPlex® 200 to quantify the fluorescence from the goat anti-mouse PE bound to beads. The fluorescence intensity is proportional the concentration of PSP94 in urine.
6. Quantitation of results were determine by a 4-PLC curve fit from

### Example 3

### Analytical Performance

### Typical Calibration Curve:

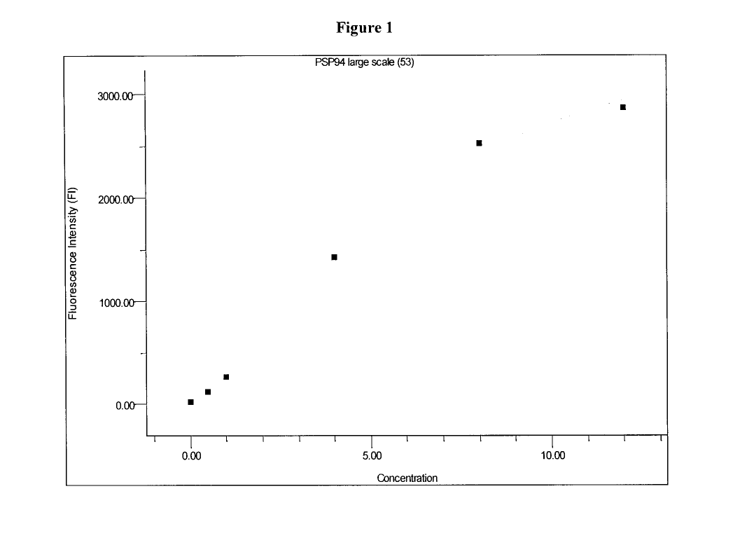

The average of duplicate readings for each calibrator control and sample are calculated using the BioPlex® 200 software to generate a 4-PLC curve fit. The zero calibrator does not need to be subtracted from the other calibrators, controls or samples for accurate results. A typical curve is shown in FIG. 1 and typical results for each calibrator are shown in Table 2.

TABLE 2
Typical Calibration Curve

| Concentration | | % | Concentration | |
|---|---|---|---|---|
| (ng/mL) | Fluorescence | CV | (ng/ml) | (Obs/Exp) * 100 |
| 12 | 2875.3 | 1.17 | 11.22 | 94 |
| 8 | 2535 | 4.18 | 8.6 | 107 |
| 4 | 1432.5 | 3.55 | 3.99 | 100 |
| 1 | 274.3 | 3.74 | 0.97 | 97 |
| 0.5 | 125.5 | 4.51 | 0.52 | 103 |
| 0 | 25.8 | 4.12 | | |

### Intra-Assay Precision:

Controls were run in duplicate or triplicate over 13 different plates for over 1 month. The Intra-assay precision for the 2 and 6 ng/mL PSP94 was less than 5% and the Inter-assay precision is less than 10%.

### Spike Recovery:

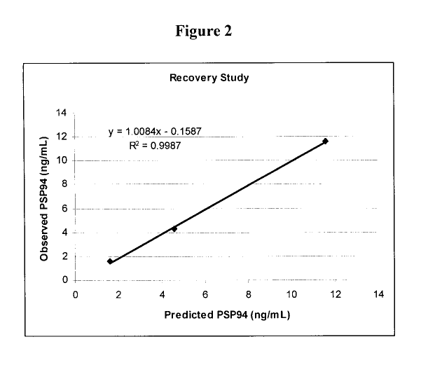

An example of recovery is provided in FIG. 2 where a sample with 1.6 ng/mL PSP94 in 1:10 diluted urine was spiked with 3 and 10 ng/mL PSP94. The % recovery was 95% for spiked samples. The slope is approximately 1.0 with a correlation coefficient of ˜1.0 indicating near perfect recovery of PSP94 in this study.

### Analytical Sensitivity:

Limit of Quantitation (LOQ) was estimated with 5 different standard assays using multiple bead preparations and reagent preparations. Specifically, the LOQ was calculated by determining the concentration of PSP94 from a 4-PLC curve fit for the

Fluorescence=mean of the zero calibrator+10*standard deviation of the zero calibrator.

The LOQ ranged from 0.10 to 0.15 ng/mL At a 1:20 dilution, the LOQ is 2.0 to 3.0 ng/mL.

### Specificity:

PSA was added at 100 ng/mL to the zero calibrator. The concentration of PSP94 was less than the LOQ. Therefore, PSA did not interfere with this assay.

Freeze-Thaw cycles:

There was no effect (total CV from 4-11% CV) of up to 4 freeze thaw cycles on the performance of PSP94 when tested on 5 fresh samples with PSP94 data ranging from 8 to 220 ng/mL.

### Example 4

### The Combination of PSP94 Results with F/T PSA for the Diagnosis of Aggressiveness of Prostate Cancer

Samples were collected from men according to Example 1. 24 hr urine samples were tested for PSP94 and the results were standardized to creatinine. Matched serum samples were then analyzed for F/T PSA. Data was then analyzed using MedCalc Software version 9.5.2.0 (2008).

We observed that both PSP94 and F/T PSA resulted in lower median values with Gleason Score ≧7. Therefore, we reasoned that multiplication of low values should increase the separation compared to the Gleason Score 6 or non cancer specimens. We also created an algorithm using Age, smoking and total PSA along with F/T PSA and PSP94 with the Split Scoring Method ( ).

To demonstrate that there is an improvement in separation of aggressive from non aggressive cancer by combining both F/T PSA and PSP94, we expected the p values of the Mann-Whitney test to be lower than the individual tests. We chose the non-parametric statistic, Mann-Whitney Test, since the data for PSP94/creatinine, F/T PSA, the combinations of PSP94/Creatinine and F/T PSA were not normally distributed. The data in Table 3 below demonstrate:
1. PSP94/Creatinine can statistically separate aggressive prostate cancer (Gleason Score ≧7) from Gleason Score 6 (p<0.05) while F/T PSA did not statistically separate aggressive prostate cancer from Gleason Score 6 (p>0.05)
2. Mann-Whitney Test p values were lowest for the combinations with PSP94 and F/T PSA as compared to either test alone. Therefore, separation between non Cancer and Gleason Score 6 from Gleason Score >=7 improves with combinations of F/T PSA and PSP94.
3.

TABLE 3

Mann-Whitney Test
Gleason Score >=7
Versus

| | Gleason | |
|---|---|---|
| Assay | Score = 6 | Non Cancer |
| PSP94/Creatinine | p = 0.0030 | p = 0.0014 |
| F/T PSA in serum | p = 0.2933 | p = 0.0044 |
| PSP94/Creatinine * F/T PSA | p = 0.0006 | p = 0.0001 |
| PSP94, F/T PSA, tPSA, | p = 0.0005 | p = 0.0001 |
| Smoking and Age | | |
| (Weighted Scoring Method) | | |

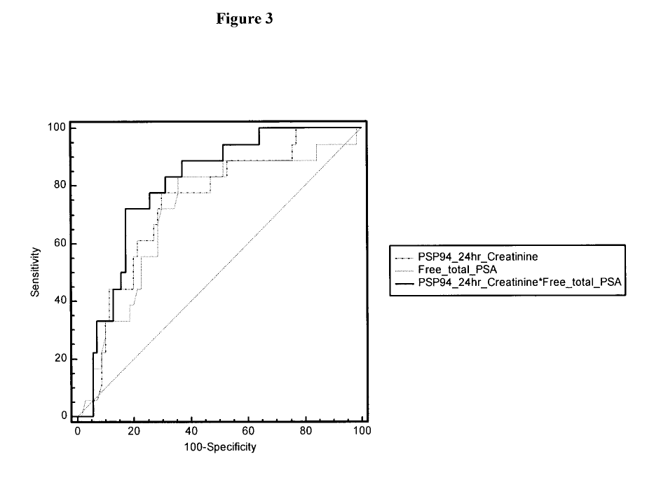

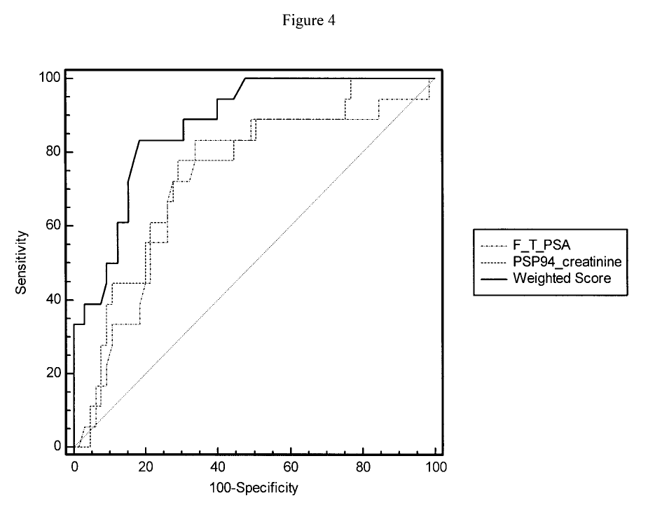

Next, we generated ROC curve for PSP94 concentration, F/T PSA and the combinations of PSP94 and F/T PSA (FIG. 3) and with algorithm with PSP94, F/T PSA, PSA, smoking and age (FIG. 4). All data were analyzed with MedCalc Software version 9.5.2.0 (2008). The ROC curve use aggressive prostate cancer (Gleason score ≧7) compared to all other samples (Gleason Score 6 and non cancer). We selected the high level sensitivity of 94% ( 17/18) or 100% ( 18/18) to demonstrate statistically significant improvement of the combinations of PSP94 and F/T PSA since we did not minimize false negative results in men with aggressive cancer. We examined the 95% confidence intervals for each individual marker and combination of biomarkers. Statistical difference is achieved when 95% CI do not overlap. The all combinations of PSP94 and F/T PSA demonstrate that there is no overlap of 95% confidence intervals between the individual tests the combination of the two tests at 94% sensitivity. Furthermore, at 100% sensitivity, F/T PSA 95% Confidence Interval does not overlap any combination of F/T PSA and PSP94 while PSP94/Creatinine 95% confidence interval did not overlap with the PSP94, F/T PSA, tPSA, age and smoking combinations. Thus, combinations with PSP94 and F/T PSA are statistically improved compared to individual biomarkers.

TABLE 4
Summary table for the ROC curve including Area under the curve (AUC),
the Specificity either 100% or 94% Sensitivity with their corresponding
95% Confidence Intervals (CI).

| | | Specificity at | | Specificity | |
|---|---|---|---|---|---|
| | | 100% | | at 94% | |
| | AUC | Sensitivity | 95% CI | Sensitivity | 95% CI |
| PSP94/ | 0.737 | 23% | 13-35%# | 24% | 15-37%* |
| Creatinine | | | | | |
| F/T PSA in | 0.720 | 2% | 0-8%* | 15% | 8-27%* |
| serum | | | | | |
| Combination | | | | | |
| PSP94/ | 0.802 | 37% | 25-50% | 51% | 38-63% |
| Creatinine × | | | | | |
| F/T PSA | | | | | |
| PSP94, F/T | 0.874 | 52% | 40-65% | 60% | 47-72% |
| PSA, tPSA, | | | | | |
| Smoking and | | | | | |
| Age | | | | | |
| (Weighted | | | | | |
| Scoring | | | | | |
| Method) | | | | | |

*No overlap of the 95% confidence intervals between individual tests and the combination of 95% confidence intervals F/T PSA and PSP94.
#No overlap of the 95% confidence intervals between PSP94/creatinine and the PSP94, F/T PSA, PSA, Smoking and Age algorithm results.

To further demonstrate statistical improvement between the individual tests and the combination of F/T PSA, we analyzed the distribution of non aggressive samples with the McNamer Test at a constant sensitivity of either 94% or 100%. Statistical improvement of the non aggressive prostate cancer specimens would indicate an improvement the specificity of the assay. The following hypotheses were tested with the McNamer Test:
The null hypothesis: The combination of F/T PSA and PSP94 has no impact on the diagnosis non aggressive prostate cancer specimens.
The alternative hypothesis: The combination of F/T PSA and PSP94 had an impact on the diagnosis non aggressive prostate cancer specimens.
   All combinations demonstrated statistical difference for the McNamer Test. This indicates that combining PSP94 and F/T PSA impacts the diagnosis of non aggressive prostate cancer specimens while diagnosing >=94% of aggressive prostate cancer specimens. Furthermore, the diagnosis of prostate cancer is improved by the combinations of PSP94, F/T PSA with other factors that can affect prostate cancer such age, smoking and tPSA.

| | | F/T PSA, PSP94, |
|---|---|---|
| | | PSA, Age, |
| | | Smoking |
| | F/T PSA * PSP94 | (Split Scoring Method) |
| PSP94 @ 94% Sensitivity | p < 0.0001 | p < 0.0002 |
| PSP94 @ 100% Sensitivity | p < 0.0039 | p < 0.0030 |
| F/T PSA @ 94% Sensitivity | p < 0.0001 | p < 0.0001 |
| F/T PSA @ 100% | p < 0.0001 | p < 0.0001 |
| Sensitivity | | |

1. A method of diagnosing prostate cancer in a subject, comprising:
(a) detecting a quantity, presence, or absence of PSP94 in a first biological sample from the subject;
(b) detecting a ratio of free to total PSA (F/T PSA) in the first biological sample or a second biological sample from the subject; and
(c) comparing the quantity, presence or absence of PSP94 and the F/T PSA as detected in steps (a) and (b) with a standard score, said standard score having information regarding F/T PSA and PSP94 levels obtained from one or more known healthy subjects, wherein a deviation in quantity, presence, or absence of PSP94 and/or F/T PSA between the quantity, presence or absence as detected in steps (a) and (b) with said standard score results in a diagnosis of prostate cancer in the subject.

2. A method of diagnosing prostate cancer in a subject, comprising:
(a) detecting a quantity, presence, or absence of PSP94 in a first biological sample from the subject;
(b) detecting a ratio of free to total PSA (F/T PSA) in the first biological sample or a second biological sample from the subject; and
(c) comparing the quantity, presence or absence of PSP94 and the F/T PSA as detected in steps (a) and (b) with a standard score, said standard score having information regarding F/T PSA and PSP94 levels obtained from one or more subjects known to have prostate cancer, wherein a similarity in quantity, presence, or absence of PSP94 and/or F/T PSA between the quantity, presence or absence as detected in steps (a) and (b) with said standard score results in a diagnosis of prostate cancer in the subject.

3. A method of diagnosing aggressive prostate cancer in a subject, comprising:
(a) detecting a quantity, presence, or absence of PSP94 in a first biological sample from the subject;
(b) detecting a ratio of free to total PSA (F/T PSA) in the first biological sample or a second biological sample from the subject; and (c) comparing the quantity, presence or absence of PSP94 and the F/T PSA as detected in steps (a) and (b) with a standard score, said standard score having information regarding F/T PSA and PSP94 levels obtained from one or more known subjects having a Gleason score of less than or
equal to 6, wherein a deviation in quantity, presence, or absence of PSP94 and/or F/T PSA between the quantity, presence or absence as detected in steps (b) and (c) with said standard score results in a diagnosis of aggressive prostate cancer in the subject.

4. A method of diagnosing aggressive prostate cancer in a subject, comprising:
(a) detecting a quantity, presence, or absence of PSP94 in a first biological sample from the subject;
(b) detecting a ratio of free to total PSA (F/T PSA) in the first biological sample or a second biological sample from the subject; and (c) comparing the quantity, presence or absence of PSP94 and the F/T PSA as detected in steps (a) and (b) with a standard score, said standard score having information regarding F/T PSA and PSP94 levels obtained from one or more subjects known to have a Gleason score of greater than or equal to 7, wherein a similarity in quantity, presence, or absence of PSP94 and/or F/T PSA between the quantity, presence or absence as detected in steps (b) and (c) with said standard score results in a diagnosis of aggressive prostate cancer in the subject.

5. A method of differential diagnosis in a subject, comprising:
(a) detecting a quantity, presence, or absence of PSP94 in a first biological sample from the subject;
(b) detecting a ratio of free to total PSA (F/T PSA) in the first biological sample or a second biological sample from the subject; and
(c) comparing the quantity, presence or absence of PSP94 and the F/T PSA as detected in steps (a) and (b) with a standard score, said standard score having information regarding F/T PSA and PSP94 levels obtained from one or more subjects known to be selected from the group consisting of (i) healthy subjects, (ii) subjects having a precancerous prostatic lesion, (iii) subjects with non-malignant disease of the prostate, (iv) subjects with localized cancer of the prostate, (v) subjects having an acute or chronic inflammation of prostatic tissue (v) subjects with metastasised cancer of the prostate, wherein a similarity or difference between the quantity, presence or absence of PSP94 and the F/T PSA in the first or the first and second biological samples and the standard score is used to determine whether the subject is (i) healthy, or has a precancerous prostatic lesion, a non-malignant disease of the prostate, a localized cancer of the prostate, an acute or chronic inflammation of prostatic tissue, or a metastasised cancer of the prostate.

6. A method of diagnosing the progression of prostate cancer in a subject, comprising: (a) detecting a quantity, presence, or absence of PSP94 in a first biological sample from the subject;
(b) detecting a ratio of free to total PSA (F/T PSA) in the first biological sample or a second biological sample from the subject; and
(c) comparing the quantity, presence or absence of PSP94 and the F/T PSA as detected in steps (a) and (b) with a Standard score, said standard score having information regarding F/T PSA and PSP94 levels obtained from the subject in the past, wherein a deviation in quantity, presence, or absence of PSP94 and/or F/T PSA between the quantity, presence or absence as detected in steps (a) and (b) with said standard score results in a indicator of the progression of the prostate cancer in the subject.

7. The method of claim 1 further comprising diagnosing whether the subject has hypertension.

8. (canceled)

9. The method of claim 1 wherein the detection of the quantity, presence or absence of PSP94 and/or F/T PSA comprises:
(a) contacting the biological sample with a biologically active surface;
(b) allowing the PSP94 and PSA within the biological sample to bind to the biologically active surface;
(c) detecting the bound PSP94 and PSA, and determining F/T PSA, using a detection method, wherein the detection method generates mass profiles of the biological sample; and
(d) transforming information obtained in (c) into a computer readable form.

10. The method of claim 1 wherein the detection of the quantity, presence or absence of PSP94 and/or F/T PSA comprises:
(a) contacting the biological sample with one or more binding molecule specific for PSP94 and PSA; and
(b) detecting the quantity, presence or absence of PSP94 and PSA, and determining F/T PSA, in the sample.

11. The method of claim 1 wherein the standard score is a database containing mass profiles from subjects whose classification is known.

12. The method of claim 11 wherein the subjects whose classification is known excludes subjects known to have hypertension.

13. The method of claim 1 wherein the PSP94 levels are standardized to the subjects' creatine levels.

14. The method of claim 1 wherein the subject is diagnosed as having aggressive prostate cancer when the quantity of PSA is determined to be between 2.5-10 ng/ml.

15. The method of claim 1 wherein the quantity, presence, or absence of PSP94 and F/T PSA are detected by mass spectrometry.

16. The method of claim 15 wherein the mass spectrometry is selected from the group consisting of matrix-assisted laser desorption ionization/time of flight (MALDI-TOF), surface enhanced laser desorption ionisation/time of flight (SELDI-TOF), liquid chromatography, MS-MS, and ESI-MS.

17. The method of claim 1 wherein the quantity, presence, or absence of PSP94 and F/T PSA are detected by utilizing an antibody specific to PSP94 or F/T PSA.

18. The method of claim 1 wherein the quantity, presence, or absence of PSP94 and F/T PSA are detected by utilizing an ELISA.

19. The method of claim 1 wherein the quantity, presence, or absence of PSP94 and F/T PSA are detected through use of an immunoassay.

20. The method of claim 1 wherein the quantity, presence, or absence of PSP94 and F/T PSA are detected or quantified by use of a biochip.

21. (canceled)

22. The method of claim 1 wherein the biological sample is selected from the group consisting of whole blood, blood serum, blood plasma, urine, semen, seminal fluid, seminal plasma, prostatic fluid, pre-ejaculatory fluid (Cowper's fluid), excreta, tears, saliva, sweat, biopsy, ascites, cerebrospinal fluid, lymph, and a biopsy sample.

23. The method of claim 1 wherein the biological sample is urine.

24. The method of claim 1 wherein the biological sample is blood.

25-27. (canceled)


SEQUENCE LISTING (2 sequences)

SEQ ID NO: 1 - protein, 114 aa, Homo sapiens
MNVLLGSVVIFATFVTLCNASCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 2 - protein, 94 aa, Homo sapiens
SCYFIPNEGVPGDSTRKCMDLKGNKHPINSEWQTDNCETCTCYETEISCCTLVSTPVGYDKDNCQRIFKKEDCKYIVVEKKDPKKTCSVSEWII